(19) United States
(12) Patent Application Publication
(10) Pub. No.: US 2020/0206368 A1
(43) Pub. Date: Jul. 2, 2020

(54) BIOLOGICS LABELING SYSTEM AND METHODS THEREOF

(71) Applicant: VisiCELL Medical Inc., San Diego, CA (US)

(72) Inventors: Mya Mya S. Thu, San Diego, CA (US); Johnny Akers, San Diego, CA (US)

(21) Appl. No.: 16/732,193

(22) Filed: Dec. 31, 2019

Related U.S. Application Data
(60) Provisional application No. 62/787,042, filed on Dec. 31, 2018.

(51) Int. Cl. A61K 49/18; A61K 9/00

(52) U.S. Cl. CPC A61K 49/1824; A61K 9/0019

(57) ABSTRACT

Nanoparticles, compositions, and methods are disclosed for manufacturing and using the nanoparticles and compositions for cellular tracking, drug encapsulation, and biologic tracking.

### PRIORITY

This application claims priority to U.S. Provisional Application No. 62/787,042 filed on Dec. 31, 2018, the entire contents of which are incorporated herein by reference.

### BACKGROUND

As the field of cell-based therapy continues to advance, there is a need for safe and effective prognostic and diagnostic tools that can be used in the clinic for non-invasive monitoring of such cell-based therapeutic products and cell-derived biologics (e.g., vesicles). Specifically, this need requires overcoming challenges associated with labelling and/or manipulating these cell-based therapeutic products and cell-derived biologics that arise due to their physiological characteristics and sensitivity to foreign materials.

A critical consideration for the development of cell-based therapeutic products and cell-derived biologics for regenerative medicine and immuno-oncology is a need to understand what happens to the cells once they have been implanted in patients to guide treatment strategies. One approach to achieving this goal is to develop cell tracking tools that are safe and possess imaging modalities that provide observation and visibility of the cells once implanted in a patient.

Common cell-based therapeutic products such as mesenchymal stem cells for regenerative medicine applications and chimeric antigen receptors (CARs) modified T lymphocyte (CAR-T) cell therapy for treating hematologic malignancies and recently, solid tumors, have advanced from laboratory to market. However, there is a critical unmet need for developing the labeling techniques for these low-phagocytic and sensitive cells in a clinically applicable way that further allows for the integration into the standard workflow of manufacturing and point of care process of these cell therapy developers. Additionally, it is critical that the technology uses and have a high performance with the standard imaging modalities and conventional imaging protocols available in most clinic for frictionless adoption and integration into the clinical imaging practice workflow.

### SUMMARY

In some aspects, the present disclosure relates to nanoparticles comprising heparin, protamine, an iron-based imaging agent, and a contrast agent, wherein the contrast agent is conjugated to the heparin and/or the protamine.

In another aspect, the present disclosure relates to a composition comprising nanoparticles comprised of heparin, protamine, and an iron-based imaging agent, and wherein the composition comprises no or substantially no unbound heparin, protamine, or iron-based imaging agent.

In some embodiments, the nanoparticle has an iron content of about 0.1 femtograms (fg) to about 1 fg. In certain embodiments, the nanoparticles are uniform in size and have an average diameter of 120 nm to 195 nm.

In another embodiments, the iron-based imaging agent is ferumoxytol.

In some embodiments, the contrast agent is a fluorescent probe, a PET agent, a CT agent, or an MRI agent.

In some embodiments, the composition comprises no or substantially no unbound heparin-protamine complexes, protamine-iron-based imaging agent complexes, or heparin-iron-based imaging agent complexes.

In some embodiments, the composition further comprises an excipient. In another embodiment, the composition is lyophilized or frozen.

In one aspect, the present disclosure relates to methods of labeling a cell or cell-derived product comprising contacting the cell or cell-derived product with a composition comprising nanoparticles comprised of heparin, protamine, and an iron-based imaging agent, wherein the composition comprises no or substantially no unbound heparin, protamine, or iron-based imaging agent.

In some embodiments, the methods further comprise applying a magnetic field while contacting the cell or cell-derived product with the composition comprising nanoparticles.

In some embodiments, the composition comprises no or substantially no unbound heparin-protamine complex, protamine-iron-based imaging agent complex, or heparin-iron-based imaging agent complex.

In certain embodiments, the methods further comprise labeling the cell in a serum-containing cell culture while contacting the cell or cell-derived product with the composition. In another embodiment, the methods further comprise labeling the cell in a serum-deprived cell culture while contacting the cell or cell-derived product with the composition.

In some embodiments, the cell or cell-derived product is labeled in less than four hours. In some embodiments, the cell or cell-derived product is labeled in greater than four hours.

In one embodiment, the cell is a mammalian cell. In some embodiments, the mammalian cell is a mouse cell, a rat cell, a canine cell, a porcine cell, a bovine cell, an equine cell, a primate cell, or a human cell. In another embodiment, the cell is a cancer cell, an immune cell, or a blood cell. In some embodiments, the cell is a stem cell, an embryonic cell, a fetal cell, or a somatic cell. In yet another embodiment, the stem cell is a mesenchymal stem cell, a pluripotent stem cell, an induced pluripotent stem cell, an adipose stem cell, a neural stem cell, or an adult stem cell.

In some embodiments, the cell-derived product is a vesicle. In some embodiments, the methods further comprise contacting the cell-derived product with a cell to label the cell with the cell-derived product.

In another embodiment, the methods further comprise tracking the cell or cell-derived product for a period of time after contacting the cell. In one embodiment, the cell tracking is ex vivo, in vitro, or in vivo tracking. In some embodiments, the tracking is performed by imaging the cell.

In some embodiments, the methods further comprise tracking the cell derived product, wherein the cell derived product is either within or outside the cell.

In one embodiment, the imaging is performed by fluorescent microscopy, MRI, FL, in vivo optical imaging, PET, SPECT, and/or ultrasound. In some embodiments, the imaging is performed at least about 1 hour, at least about 2 hours, at least about 6 hours, at least about 12 hours, at least about 24 hours, at least about 2 days, at least about one week, at least about a month, at least about 3 months, at least about 6 months, at least about 1 year, at least about 2 years, at least about 3 years, at least about 4 years, at least about 5 years, or more, after the cell is contacted with the nanoparticle or the composition.

In some embodiments, the methods are for in vitro or in vivo labeling of the cell.

In another embodiment, the methods further comprise administering the composition to a subject. In some embodiments, the subject is a mammal. In some embodiments, the mammal is a human. In yet another embodiment, the composition is administered intravenously, intra-arterial, direct tissue injection, and/or intratumorally.

In some embodiments, the methods further comprise encapsulating a nucleic acid in the nanoparticle before contacting the composition comprising nanoparticles with the cell or cell-derived product.

Another aspect of the present disclosure relates to methods of preparing magnetically separated nanoparticles, the methods comprising: (a) combining heparin, protamine, and an iron-based imaging agent, wherein at least a portion of the heparin, protamine, and an iron-based imaging agent to form nanoparticles comprising heparin, protamine, and an iron-based imaging agent; and (b) exposing the nanoparticles to a magnetic field to separate the nanoparticles based on a magnetic moment of the nanoparticles.

In some embodiments, exposing the solution to the magnetic field further separates unbound protamine, heparin, or iron-based labeling agent that did not complex to form the nanoparticles.

In some embodiments, the nanoparticles are further separated based on size.

In another embodiment, exposing the solution to a magnetic field comprises applying a magnet to the solution. In some embodiments, the magnet has a magnetic field strength of about 300 mT to about 600 mT.

In some embodiments, the magnetic field is uniform and applied perpendicular to the nanoparticles. In another embodiment, the magnetic field is non-uniform and directionally changing.

A method of labeling a cell from a cell-derived product, the method comprising: (a) contacting a first cell with a composition comprising nanoparticles comprised of heparin, protamine, and an iron-based imaging agent, wherein the contacting step labels the cell derived product within the cell; (b) removing the labeled cell derived product from the cell; and (c) contacting a second cell with the cell derived product, wherein the second cell uptakes the labeled cell derived product. In some embodiments, the methods further comprise directly tracking the labeled cell derived product from the first cell. In some embodiments, the methods further comprise tracking the second cell.

These and other features, aspects and advantages of the present teachings will become better understood with reference to the following description, examples and appended claims.

### DRAWINGS

This application contains at least one drawing executed in color. Copies of this application with color drawing(s) will be provided by the Office upon request and payment of the necessary fees.

FIG. 1A is a representative graph demonstrating the size distribution of standard HPF nanoparticles that have not been prepared by the magnetic separation process of the present disclosure.

FIGS. 1B-1C are representative graphs demonstrating the size distribution of VMI NCE 1 nanoparticles synthesized according to the magnetic separation process of the present disclosure.

FIG. 1D are representative images that show that VMI NCE 1 nanoparticles synthesized according to the magnetic separation process of the present disclosure do not have residual unbound ferumoxytol whereas standard HPF nanoparticles do.

FIG. 2A-2B are representative images of Prussian blue staining of cells showing the iron loading of cells labeled with nanoparticles of the present disclosure.

FIGS. 3A-3B are representative graphs showing an increase in cell viability upon labeling cells with VMI NCE 1 nanoparticles of the present disclosure relative to standard HPF nanoparticles.

FIG. 3C shows by TEM analysis that VMI NCE 1 nanoparticles synthesized according to the magnetic separation process of the present disclosure hold more electron dense magnetically active MRI responsive iron agents relative to standard HPF nanoparticles.

FIGS. 4A and 4B are representative images of Prussian blue staining of cells labeled with nanoparticles of the present disclosure at various concentrations.

FIG. 5A are representative images of Prussian blue staining of cells emphasizing the difference in cell labeling efficiency for VMI NCE 1 nanoparticles prepared by magnetic separation according to the methods of the present disclosure.

FIG. 5B is a representative graph showing the viability of cells labeled with the particles of the present disclosure.

FIG. 5C-5D are representative graphs showing the particle size distribution of the standard HPF and VMI NCE 1 prepared by magnetic separation according to the methods of the present disclosure.

FIG. 6 are representative images of Prussian blue staining of cells showing that the concentration of the VMI NCE 1 nanoparticles of the present disclosure can be increased 10-fold without decreasing cell viability as is needed to label the non-phagocytic cells or low-phagocytic cells.

FIG. 7 are representative images of Prussian blue staining of cells showing that the cells can be labeled with the VMI NCE 1 nanoparticles of the present disclosure under serum-containing conditions without the need for serum starvation.

FIGS. 8A-8B are representative images showing the ability to label cells using the magnetic labeling system of the present disclosure in less than 3 hours under serum starvation conditions as well as under serum containing conditions.

FIG. 9A-9C are representative images showing the Prussian blue staining and the fluorescent activity of the dual probe VMI NCE 1 nanoparticles of the present disclosure.

FIG. 10A are representative graphs showing an increase in cell viability upon labeling cells with dual-probe VMI NCE 1 nanoparticles of the present disclosure relative to standard dual-probe HPF particles.

FIG. 10B-10C are representative graphs demonstrating the average diameter of dual probe VMI NCE 1 nanoparticles synthesized according to the methods of the present disclosure. In FIG. 10C, the dual probe VMI NCE 1 residual curve indicates the unbound residual components that are present in excess and toxic to cells whereas the dual probe VMI NCE 1 curve shows the removal of the unbound toxic material.

FIG. 11 are representative images showing the use of a magnetic labeling system to facilitate the uptake of the nanoparticles of the present disclosure to efficiently and effectively label cells having a desired iron load.

FIGS. 12A and 12B are representative images of the MRI signal of cells labeled with nanoparticles of the present disclosure in a 7T small animal scanner using a conventional T2-weighted MRI and in a 3T clinical scanner using a conventional MRI T2*-weighted MRI.

FIG. 13 are representative color maps of different MR parameters (e.g., (a) QSM map (b) T₁ map (c) T₂ map (d) T₂* map) (top images) and their correlation fittings between the density and the MR parameters (lower images) of nanoparticles of the present disclosure in a 3T clinical scanner.

FIG. 14 shows representative TEM images of vesicles labeled with nanoparticles of the present disclosure.

FIG. 15A-15B are representative images of vesicles labeled with nanoparticles of the present disclosure in a 7T small animal scanner using a conventional T2 and T2* weighted MRI.

FIG. 16 are representative images of cells labeled with nanoparticles of the present disclosure embedded with nucleic acids.

FIG. 17-19 are schematic representations of protocols disclosed and described herein.

### DETAILED DESCRIPTION

Provided herein are nanoparticles, compositions, and methods for manufacturing and using the nanoparticles and compositions for cellular tracking, drug encapsulation, and biologic tracking.

There is a need to develop techniques for cell-labeling of many cell types including low-phagocytic and non-phagocytic cells via non-invasive magnetic resonance imaging (MRI) detection. The present disclosure describes a magnetic separation process that synthesizes and produces a population of heparin (H): protamine (P): feraheme (F) particles (HPF particles) with enriched iron content and increased magnetic properties (VMI NCE 1 nanoparticles) in order to improve the MRI signal for use with conventional MRI sequences available in most clinical settings. Significantly, the results from this study provide magnetically enriched HPF particles having a high cell-labeling efficiency and a favorable safety profile. The results further demonstrate that the HPF particles produced and manufactured using the methods disclosed herein can be used in cell labeling and tracking. The present disclosure also provides a magnetic labeling system that allows for a shortened (e.g., less than 3 hours) labeling of cells in both serum and serum starved conditions.

After reading this description it will become apparent to one skilled in the art how to implement the invention in various alternative embodiments and alternative applications. However, all the various embodiments of the present invention will not be described herein. It will be understood that the embodiments presented here are presented by way of example only, and not limitation. As such, this detailed description of various alternative embodiments should not be construed to limit the scope or breadth of the present invention as set forth below.

The detailed description is divided into various sections only for the reader's convenience and disclosure found in any section may be combined with that in another section. Titles or subtitles may be used in the specification for the convenience of a reader, which are not intended to influence the scope of the present disclosure.

Unless the context indicates otherwise, it is specifically intended that the various features of the invention described herein can be used in any combination.

### Definitions

All numerical designations are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about.” It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth. It also is to be understood, although not always explicitly stated, that the reagents described herein are merely exemplary and that equivalents of such are known in the art.

The term “about,” as used herein when referring to a measurable value such as an amount or concentration and the like, is meant to encompass variations of 20%, 10%, 5%, 1%, 0.5%, or even 0.1% of the specified amount.

The phrase “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit:risk ratio.

### Nanoparticles

Aspects of the present disclosure relate to nanoparticles comprising heparin, protamine, and an iron-based imaging agent. In some embodiments, the nanoparticles further comprise an additional imaging agents and/or nucleic acid.

In some embodiments, the nanoparticles of the present disclosure comprise heparin. Heparin can be either synthetic or a biologically sourced, has been incorporated into medicinal and biological applications, including nanoparticles, to improve the overall biocompatibility. In some embodiments, the heparin is in the form of a salt. For example, in some embodiments, the heparin is in the form of a sodium, calcium, potassium, or lithium salt. In some embodiments, the heparin is heparan sulfate. In some embodiments, the heparin is heparin sodium. In some embodiments, heparin sodium is more suitable as compared to heparan sulfate due to the number of sulfate groups present in heparan sulfate, which gives rise to higher anionic properties of the heparin. In some embodiments, heparin has a low molecular weight.

In some embodiments, the heparin may be used (synthetic or biologically sourced from any manufacturers) for complexing with protamine and an iron-based imaging agent without the need to know the exact molecular weight and chain length of the heparin. This highly variable due to its biologically sourced nature and which are impossible to obtain without significant cost and time. In some embodiments, the ratio agnostic due to an ability to extract the desired VMI NCE 1 particles after the standard HPF particles are formed at different starting ratios. In some embodiments, the total yield of the desired VMI NCE1 particles may be different and thus there are recommended ranges disclosed herein.

In some embodiments, the heparin is provided in an amount of about 0.01 U/ml to about 10 U/ml. In some embodiments, the heparin is in an amount of about 0.25 U/ml, about 0.5 U/ml, about 0.75 U/ml, about 1 U/ml, about 1.25 U/ml, about 1.5 U/ml, about 1.75 U/ml, about 2 U/ml, about 2.25 U/ml, about 2.5 U/ml, about 2.75 U/ml, about 3 U/ml, about 3.25 U/ml, about 3.5 U/ml, about 3.75 U/ml, about 4 U/ml, about 4.25 U/ml, about 4.5 U/ml, about 4.75 U/ml, about 5 U/ml, about 5.25 U/ml, 5.5 U/ml, about 5.75 U/ml, about 6 U/ml, about 6.25 U/ml, about 6.5 U/ml, about 6.75 U/ml, about 7 U/ml, about 7.25 U/ml, about 7.5 U/ml, about 7.75 U/ml, about 8 U/ml, 8.25 U/ml, about 8.5 U/ml, about 8.75 U/ml, about 9 U/ml, about 9.25 U/ml, about 9.5 U/ml, about 9.75 U/ml, or about 10 U/ml.

In some embodiments, the nanoparticles of the present disclosure further comprise protamine. Protamine either be synthetic or biologically sourced and is used in therapies to neutralize the strongly negatively charged heparin by complexing with it to form a stable salt. In some embodiments, the protamine is obtained from the sperm of salmon or other fish species. In some embodiments, the protamine is in the form of a sulfate, phosphate, or hydrochloride salt. In some embodiments, the protamine is sulfate salt from salmon. In some embodiments, the protamine is protamine sulfate. In some embodiments, protamine has a low molecular weight.

In some embodiments, the protamine may be used (synthetic or biologically sourced from any manufacturers) for complexing with heparin and an iron-based agent without the need to know the protamine's exact molecular weight and chain length, which are highly variable due to its biologically sourced nature and which are impossible to obtain without significant cost and time. In some embodiments, the ratio is agnostic due to the ability to extract the desired nanoparticles after the standard particles are formed at different starting ratios. It is worth nothing that the total yield of the desired nanoparticles may be different and thus there are recommended ranges disclosed herein.

In some embodiments, the protamine is provided in an amount of about 1 μg/ml to about 200 μg/ml. In some embodiments, the protamine is provided in an amount of about 10 μg/ml, about 20 μg/ml, about 30 μg/ml, about 40 μg/ml, about 50 μg/ml, about 60 μg/ml, about 70 μg/ml, about 80 μg/ml, about 90 μg/ml, about 100 μg/ml, about 120, μg/ml, about 130 μg/ml, about 140 μg/ml, about 150 μg/ml, about 160 μg/ml, about 170 μg/ml, about 180 μg/ml, about 190 μg/ml, or about 200 μg/ml.

In some embodiments, the nanoparticles of the present disclosure further comprise an imaging agent. The imaging agent functions to allow visualization of the nanoparticles themselves or once the nanoparticles are incorporated with a biological material, for example a cell, via an imaging modality. In some embodiments, the imaging agent is a superparamagnetic metal complex. In some embodiments, the imaging agent is an iron-based agent. In some embodiments, the imaging agent is any superparamagnetic iron containing compound. In some embodiments, the imaging agent is a superparamagnetic iron oxide. In other embodiments, the imaging agent is an ultrasmall superparamagnetic iron oxide. In some embodiments, the same iron-based imaging agent is used for magnetic separation and production of the VMI NCE 1 nanoparticles. Non-limiting examples of suitable imaging agents include ferumoxytol (Feraheme), ferumoxides, ferucarbotran, ferumoxtran-10 and iron oxides

In some embodiments, the iron-based agent is provided in an amount of about 1 μg/ml to about 800 μg/ml. In some embodiments, the labeling agent is provided in an amount of about 10 μg/ml, about 20 μg/ml, about 30 μg/ml, about 40 μg/ml, about 50 μg/ml, about 60 μg/ml, about 70 μg/ml, about 80 μg/ml, about 90 μg/ml, about 100 μg/ml, about 120, μg/ml, about 130 μg/ml, about 140 μg/ml, about 150 μg/ml, about 160 μg/ml, about 170 μg/ml, about 180 μg/ml, about 190 μg/ml, about 200 μg/ml, about 210 μg/ml, about 220 μg/ml, about 230 μg/ml, about 240 μg/ml, about 250 μg/ml, about 260 μg/ml, about 270 μg/ml, about 280 μg/ml, about 290 μg/ml, about 300 μg/ml, about 310 μg/ml, about 320 μg/ml, about 330 μg/ml, about 340 μg/ml, about 350 μg/ml, about 360 μg/ml, about 370 μg/ml, about 380 μg/ml, about 390 μg/ml, about 400 μg/ml, about 410 μg/ml, about 420 μg/ml, about 430 μg/ml, about 440 μg/ml, about 450 μg/ml, about 460 μg/ml, about 470 μg/ml, about 480 μg/ml, about 490 μg/ml, about 500 μg/ml, about 510 μg/ml, about 520 μg/ml, about 530 μg/ml, about 540 μg/ml, about 550 μg/ml, about 560 μg/ml, about 570 μg/ml, about 580 μg/ml, about 590 μg/ml, about 600 μg/ml, about 610 μg/ml, about 620 μg/ml, about 630 μg/ml, about 640 μg/ml, about 650 μg/ml, about 660 μg/ml, about 670 μg/ml, about 680 μg/ml, about 690 μg/ml, about 700 μg/ml, about 710 μg/ml, about 720 μg/ml, about 730 μg/ml, about 740 μg/ml, about 750 μg/ml, about 760 μg/ml, about 770 μg/ml, about 780 μg/ml, about 790 μg/ml, or about 800 μg/ml.

One of skill in the art would understand that different amounts of each of the heparin, protamine, and an iron-based imaging agent will depend, in part, on the desired concentration and/or amount of nanoparticles to be produced. In some embodiments, the nanoparticles comprise heparin, protamine, and ferumoxytol in different ratios. In some embodiments, the heparin is present in an amount of about 2 U/ml, the protamine is in an amount of about 60 μg/ml, and the ferumoxytol is in an amount of about 50 μg/ml. In some embodiments, the heparin is present in an amount of about 2 U/ml, the protamine is in an amount of about 40 μg/ml, and the ferumoxytol is in an amount of about 100 μg/ml.

In some embodiments, the heparin is present in an amount of about 0.01 to about 10 U/ml, the protamine is in an amount of about 1 to about 200 μg/ml, and the ferumoxytol is in an amount of about 1 μg/ml to about 800 μg/ml.

In some embodiments, the VMI NCE 1 nanoparticles further comprise an imaging contrast agent. In some embodiments, the imaging contrast agent is encapsulated in the nanoparticle or conjugated to one component of the nanoparticle, for example, protamine and/or heparin or conjugated to the nanoparticle. Non-limiting examples of suitable imaging contrast agents include a fluorescent probe, a positron emission tomography (PET) agent, a computerized topography (CT) agent (ex. Gold), an ultrasound agent, or a magnetic resonance imaging (MRI) agent.

In some embodiments, the fluorescent probe is indocyanine green (ICG), fluorescein, photofrin, or 5-aminolevulinic acid (ALA).

In some embodiments, the PET agent comprises a radionucleotide (e.g., radiotracer) selected from the group consisting of carbon-11, nitrogen-13, oxygen-15, fluorine-18, gallium-68, zirconium-89, fluorine-18, and rubidium-82. In some embodiments, the PET agent is fluorodeoxyglucose (FDG). In some embodiments, the PET agent is zirconium-89.

In some embodiments, the CT agent is an iodine or barium based compound or gold.

In some embodiments, the ultrasound agent is fluorinated hydrocarbon such as perfluorocarbon or octafluoropropane.

In some embodiments, the MRI agent is a gadolinium agent, an iron oxide agent, an iron platinum agent, or a magnesium agent.

In some embodiments, the nanoparticles comprise a dual-labeling agent for multi-modal imaging. No single imaging modality allows overall structural, functional, and molecular information because each imaging modality has its own unique characteristics. However, the combination of two imaging modalities can combine the unique characteristics of each imaging modality and thereby, improve the diagnostic and therapeutic monitoring abilities of the nanoparticles. In some embodiments, the nanoparticles further comprise an additional imaging agent. In some embodiments, the additional imaging agent is a fluorescent dye selected from the group consisting of fluorescein-5-isothiocyanate (FITC), Cy5, IRDye560, Cy7, ZW800, IRDye 800, ICG, and CH1055. In some embodiments, the additional imaging agents allows monitoring of the nanoparticle via fluorescence microscopy.

In some embodiments, the nanoparticle further comprises a gene-editing tool, for example, a nucleic acid-based molecule(s). In some embodiments, the agent is encapsulated or conjugated to the nanoparticle. In some embodiments, encapsulating or conjugating the gene editing tool to the nanoparticle enables cellular uptake of the gene editing tool. In some embodiments, the gene-editing tool is an RNA, for example, siRNA, miRNA, and mRNA. In some embodiments, the gene editing tool is an episomal in nature. In some embodiments, the gene editing tool is a DNA, for example, plasmid, double-stranded DNA, a single-stranded DNA. In some embodiments, the gene editing tool is plasmid vector, for example, CRISPR-Cas9 system. In some embodiments, the gene editing tool is a genetic material for expression of certain proteins or receptors, for example, Chimeric Antigen Receptor (CAR) for cells such as T Lymphocytes and Natural Killer cells.

In some embodiments, the nanoparticles are uniform in size or substantially uniform in size. In some embodiments, the nanoparticles have an average diameter about 125 nm to about 195 nm, and the average diameter is no smaller than 95 nm. For example, in some embodiments, the nanoparticles have an average diameter of about 125 nm to about 130 nm, about 125 nm to about 180 nm, about 130 nm to about 160 nm, about 140 nm to about 160 nm, about 140 nm to about 190 nm, about 140 nm to about 150 nm, about 125 nm to about 145 nm, about 130 nm to about 140 nm, about 130 nm to about 150 nm, about 140 nm to about 160 nm, about 140 nm to about 170 nm, about 160 nm to about 175 nm, or about 130 nm to about 180 nm. In some embodiments, the nanoparticles have an average particle size of no more than about 130 nm, no more than about 135 nm, no more than about 140 nm, no more than about 145 nm, no more than about 150 nm, no more than about 155 nm, no more than about 160 nm, no more than about 165 nm, not more than about 170 nm, not more than about 175 nm, no more than about 180 nm, no more than about 190 nm, or no more than about 195 nm.

In some embodiments, the nanoparticles having an average diameter about 125 nm to about 195 nm, and the average diameter is no smaller than 95 nm comprise at least about 0.5×10⁹ particles/mL, about 1×10⁹ particles/mL, about 2×10⁹ particles/mL, about 3×10⁹ particles/mL, about 4×10⁹ particles/mL, about 5×10⁹ particles/mL, about 6×10⁹ particles/mL, about 7×10⁹ particles/mL, about 8×10⁹ particles/mL, or about 9×10⁹ particles/mL in a synthesized sample.

In some embodiments, the nanoparticle is a magnetic nanoparticle comprising many iron-based nanoparticles with a single or a few magnetic domains. Magnetic domain is a region of the nanoparticles in which the magnetic moments of the iron atoms are aligned with one another. In some embodiments, the nanoparticle has more than 1000 magnetic domains. In some embodiments, the nanoparticles have more than about 1500 magnetic domains, more than about 2000 magnetic domains, more than about 2500 magnetic domains, more than about 3000 magnetic domains, more than about 3500 magnetic domains, more than about 4000 magnetic domains, more than about 4500 magnetic domains, or more than about 5000 magnetic domains.

In some embodiments, the iron content of the nanoparticle is greater than about 0.1 fg (femtogram). In some embodiments, the iron content is greater than about 0.15 fg, about 0.20 fg, about 0.25 fg, about 0.30 fg, about 0.35 fg, about 0.40 fg, about 0.45 fg, about 0.50 fg, about 0.55 fg, about 0.60 fg, about 0.65 fg, about 0.70 fg, about 0.75 fg, about 0.80 fg, about 0.85 fg, about 0.90 fg, about 0.95, or about 1 fg. In some embodiments, the iron content of the nanoparticles is between about 0.1 fg to about 1 fg. For example, in some embodiments, the iron content of the nanoparticles is between about 0.1 fg to about 0.50 fg, about 0.1 fg to about 0.60 fg, about 0.50 fg to about 1 fg, about 0.3 fg to about 0.80 fg, about 0.4 fg to about 0.70 fg, about 0.80 fg to about 1 fg, about 0.1 fg to about 0.4 fg, about 0.1 fg to about 0.65 fg, or about 0.15 fg to about 0.35 fg.

In some embodiments, the nanoparticle has the magnetic properties of r1, r2 and r2* that are about 3 to 10×'s greater than the iron-imaging agent (i.e., free iron-imaging agent not incorporated into the nanoparticle). For example, in some embodiments, the nanoparticles encapsulated with ferumoxytol have the magnetic properties of r1, r2 and r2* about 3 to ten times those of the ferumoxytol nanoparticles by themselves (r1=7.5 and r2=92 mM⁻¹ sec⁻¹ at 3T and r1=2 and r2=95 mM⁻¹ sec⁻¹ at 7T in water at 37° C.).

### Nanoparticle Compositions

Aspects of the present disclosure relate to compositions comprising nanoparticles.

In some embodiments, the nanoparticles comprising heparin, protamine, and an imaging agent can be formulated as a composition. In some embodiments, the imaging agent is ferumoxytol.

In some embodiments, the compositions further comprise an excipient, diluent or carrier. In some embodiments, the compositions further comprise a pharmaceutically acceptable excipient, diluent or carrier.

In some embodiments, the composition may comprise a pharmaceutically acceptable excipient, a pharmaceutically acceptable salt, diluent, carrier, vehicle and such other inactive agents. Vehicles and excipients commonly employed in pharmaceutical preparations include, for example, talc, gum Arabic, lactose, starch, magnesium stearate, cocoa butter, aqueous or non-aqueous solvents, oils, paraffin derivatives, glycols, etc. Solutions can be prepared using water or physiologically compatible organic solvents such as ethanol, 1,2-propylene glycol, polyglycols, dimethylsulfoxide, fatty alcohols, triglycerides, partial esters of glycerine and the like. Parenteral compositions may be prepared using conventional techniques that may include sterile isotonic saline, water, 1,3-butanediol, ethanol, 1,2-propylene glycol, polyglycols mixed with water, Ringer's solution, etc. In one aspect, a coloring agent is added to facilitate in locating and properly placing the composition to the intended site.

In some embodiments, the compositions further comprise a stabilizer and/or a preservative. Non-limiting examples of preservatives include methyl-, ethyl-, propyl-parabens, sodium benzoate, benzoic acid, sorbic acid, potassium sorbate, propionic acid, benzalkonium chloride, benzyl alcohol, thimerosal, phenylmercurate salts, chlorhexidine, phenol, 3-cresol, quaternary ammonium compounds (QACs), chlorbutanol, 2-ethoxyethanol, and imidurea.

In some embodiments, the composition can comprise a physiological salt, such as a sodium salt to control tonicity. In some embodiments, the physiological salt is sodium chloride (NaCl), potassium chloride, potassium dihydrogen phosphate, disodium phosphate dehydrate, magnesium chloride and calcium chloride. In some embodiments, the physiological salt is present in amount between about 1 mg/ml and about 20 mg/ml. For example, in some embodiments the physiological salt is present in an amount of about 1 mg/ml to about 5 mg/ml, about 1 mg/ml to about 10 mg/ml, about 5 mg/ml to about 20 mg/ml, about 2 mg/ml to about 5 mg/ml, about 6 mg/ml to about 10 mg/ml, about 7 mg/ml to about 20 mg/ml, about 3 mg/ml to about 18 mg/ml, about 4 mg/ml to about 10 mg/ml, about 6 mg/ml to about 15 mg/ml, or about 5 mg/ml to about 15 mg/ml.

In some embodiments, the compositions further comprise one or more buffers. Non-limiting examples of buffers include a phosphate buffer, a Tris buffer, a borate buffer, a succinate buffer, a histidine buffer, or a citrate buffer. In some embodiments, the compositions will comprise a buffer, wherein the buffer has a concentration of about 5 mM to about 20 mM range. For example, in some embodiments, the buffer has a concentration of about 5 mM to about 10 mM, about 10 mM to about 20 mM, about 5 mM to about 15 mM, about 10 mM to about 15 mM, or about 8 mM to about 20 mM. In some embodiments, the pH of a composition will be between about 5 to about 8. For example, in some embodiments, the pH of the compositions will be between about 5 to about 7.5, about 6 to about 8, about 6.5 to about 7.5, or between 7.0 and 7.8.

In some embodiments, the composition may further comprise a cryoprotectant agent. The cryoprotectant protects biological tissue from freezing damage due to ice formation. Non-limiting examples of cryoprotectant agents include a glycol (e.g., ethylene glycol, propylene glycol, and glycerol), dimethyl sulfoxide (DMSO), formamide, sucrose, trehalose, dextrose, and any combinations thereof.

In some embodiments, the composition comprises no or substantially no unbound heparin. In some embodiments, the composition comprises no more than about 10% unbound heparin, about 9% unbound heparin, about 8% unbound heparin, about 7% unbound heparin, about 6% unbound heparin, about 5% unbound heparin, about 4% unbound heparin, about 3% unbound heparin, about 2% unbound heparin, about 1% unbound heparin, about 0.5% unbound heparin, about 0.1% unbound heparin, about 0.05% unbound heparin, about 0.01% unbound heparin, or less of the total heparin in the composition. In some embodiments, the low percentage of unbound heparin decreases the toxicity of the composition.

In some embodiments, the composition comprises no or substantially no unbound protamine. In some embodiments, the composition comprises no more than about 10% unbound protamine, about 9% unbound protamine, about 8% unbound protamine, about 7% unbound protamine, about 6% unbound protamine, about 5% unbound protamine, about 4% unbound protamine, about 3% unbound protamine, about 2% unbound protamine, about 1% unbound protamine, about 0.5% unbound protamine, about 0.1% unbound protamine, about 0.05% unbound protamine, about 0.01% unbound protamine, or less of the total protamine in the composition. In some embodiments, the low percentage of unbound protamine decreases the toxicity of the composition.

In some embodiments, the composition comprises no or substantially no unbound ferumoxytol. In some embodiments, the composition comprises no more than about 10% unbound ferumoxytol, about 9% unbound ferumoxytol, about 8% unbound ferumoxytol, about 7% unbound ferumoxytol, about 6% unbound ferumoxytol, about 5% unbound ferumoxytol, about 4% unbound ferumoxytol, about 3% unbound ferumoxytol, about 2% unbound ferumoxytol, about 1% unbound ferumoxytol, about 0.5% unbound ferumoxytol, about 0.1% unbound ferumoxytol, about 0.05% unbound ferumoxytol, about 0.01% unbound ferumoxytol, or less of the total ferumoxytol in the composition. In some embodiments, the low percentage of unbound ferumoxytol decreases the toxicity of the composition.

In some embodiments, the compositions comprise no or substantially no unbound protamine and heparin. In some embodiments, the composition comprises no more than about 10% unbound protamine and heparin, about 9% unbound protamine and heparin, about 8% unbound protamine and heparin, about 7% unbound protamine and heparin, about 6% unbound protamine and heparin, about 5% unbound protamine and heparin, about 4% unbound protamine and heparin, about 3% unbound protamine and heparin, about 2% unbound protamine and heparin, about 1% unbound protamine and heparin, about 0.5% unbound protamine and heparin, about 0.1% unbound protamine and heparin, about 0.05% unbound protamine and heparin, about 0.01% unbound protamine and heparin, or less of the total protamine and heparin in the composition. In some embodiments, the low percentage of unbound heparin-protamine complexes decreases the toxicity of the composition.

In some embodiments, moisture is removed from the composition. In some embodiments, the composition is lyophilized, desiccated, cryodesiccated, and/or dehydrated to remove moisture.

In one embodiment, less than about 5% of the nanoparticles within the composition have a particle size greater than 200 nm, greater than 300 nm, greater than 400 nm, greater than 500 nm, greater than 600 nm, greater than 700 nm, or greater than 800 nm. Less than about 0.01% of the nanoparticles within the composition have a particle size greater than 200 nm, greater than 300 nm, greater than 400 nm, greater than 500 nm, greater than 600 nm, greater than 700 nm, or greater than 800 nm. In another embodiment, less than about 0.5% of the nanoparticles within the composition have a particle size greater than 200 nm, greater than 300 nm, greater than 400 nm, greater than 500 nm, greater than 600 nm, greater than 700 nm, or greater than 800 nm. In some embodiments, less than about 0.05% of the nanoparticles within the composition have a particle size greater than 200 nm, greater than 300 nm, greater than 400 nm, greater than 500 nm, greater than 600 nm, greater than 700 nm, or greater than 800 nm.

In certain embodiments, the nanoparticles within the composition have an average diameter of about 120 nm to about 190 nm. In some embodiments, the nanoparticles within the composition have an average diameter about 125 nm to about 195 nm, and the average diameter is no smaller than 95 nm. For example, in some embodiments, the nanoparticles have an average diameter of about 125 nm to about 130 nm, about 125 nm to about 180 nm, about 130 nm to about 160 nm, about 140 nm to about 160 nm, about 140 nm to about 190 nm, about 140 nm to about 150 nm, about 125 nm to about 145 nm, about 130 nm to about 140 nm, about 130 nm to about 150 nm, about 140 nm to about 160 nm, about 140 nm to about 170 nm, about 160 nm to about 175 nm, or about 130 nm to about 180 nm. In some embodiments, the nanoparticles have an average particle size of no more than about 130 nm, no more than about 135 nm, no more than about 140 nm, no more than about 145 nm, no more than about 150 nm, no more than about 155 nm, no more than about 160 nm, no more than about 165 nm, not more than about 170 nm, not more than about 175 nm, no more than about 180 nm, no more than about 190 nm, or no more than about 195 nm.

### Kits

Aspects of the present disclosure relate to kits comprising nanoparticles or compositions comprising nanoparticles.

In some embodiments, the kit comprises the nanoparticles or a composition comprising the nanoparticles and instructions that describe a protocol for preparing the nanoparticles. In some embodiments, the kit further comprises instructions for administering the nanoparticles or compositions comprising nanoparticles to a cell or a subject in need thereof.

In some embodiments, the kit comprises nanoparticles and compositions comprising nanoparticles, wherein the nanoparticles are comprised of heparin, protamine, and an imaging agent. In some embodiments, the imaging agent is ferumoxytol.

In some embodiments, the kit comprises dual-probe nanoparticles or a composition comprising the dual-probe particles and instructions that describe a protocol for preparing the dual-probe particles. In some embodiments, the kit further comprises instructions for administering the dual-probe nanoparticles or compositions comprising the dual-probe nanoparticles to a cell or a subject in need thereof.

In some embodiments, the kit comprises a magnetic labeling system for labeling of the cells with the nanoparticles or a composition comprising the nanoparticles in less than 3 hours and instructions that describe a protocol for preparing the nanoparticles and use with the magnetic labeling system. In some embodiments, the kit further comprises instructions for administering the nanoparticles or compositions comprising the nanoparticles to a cell or a subject in need thereof.

### Methods of Production

Aspects of the present disclosure relate to methods of preparing magnetically separated nanoparticles.

In some embodiments, the methods include preparing magnetically separated nanoparticles comprising heparin, protamine, and an iron-based imaging agent. In some embodiments, the methods comprise (a) combining heparin, protamine, and an iron-based imaging agent, wherein at least a portion of the heparin, protamine, and an iron-based imaging agent complex to form nanoparticles comprising heparin, protamine and an iron-based imaging agent; and (b) exposing the nanoparticles to a magnetic field to separate the nanoparticles based on a magnetic moment of the nanoparticles. In some embodiments, the separation is based on the iron content of the nanoparticles which in turn results in the separation of nanoparticles based on size and magnetic moment.

In some embodiments, the methods further comprise separating the nanoparticles from unbound material. In some embodiments, the heparin, protamine, and an imaging agent are combined in a medium, wherein at least a portion of the heparin, protamine, and an iron-based imaging agent complex to form nanoparticles comprising heparin, protamine, and an iron-based imaging agent. In some embodiments, the medium is a buffer, serum, a nutrient enriched broth, or a basal medium. Unbound material includes any compound, substance, debris, or by product that did not complex to form the nanoparticles comprising heparin, protamine, and an iron-based imaging agent. In some embodiments, the unbound material is heparin, protamine, ferumoxytol, and/or heparin-protamine complexes. In some embodiments, the methods further comprise separating the nanoparticles from unbound heparin, unbound protamine, unbound ferumoxytol and/or unbound heparin-protamine complexes. Application of the magnetic field selectively separates the nanoparticles comprising heparin, protamine, and an iron-based imaging agent from unbound heparin, unbound protamine, unbound ferumoxytol and/or unbound heparin-protamine complexes based on the iron content of the nanoparticles and thus the magnetic properties of the nanoparticles. In some embodiments, application of the magnetic field excludes unbound heparin, unbound protamine, unbound ferumoxytol and/or unbound heparin-protamine complexes to allow for the selective separation of the nanoparticles.

In some embodiments, the methods comprise (a) combining heparin, protamine, and an iron-based imaging agent, wherein at least a portion of the heparin, protamine, and an iron-based imaging agent complex to form nanoparticles comprising heparin protamine and an iron-based imaging agent; and (b) exposing the nanoparticles to a magnetic field to separate unbound protamine and/or heparin that did not complex to form the nanoparticles.

In some embodiments, the solution is exposed to the magnetic field 1 time, 2 times, 3 times, 4 times, 5 times, 6 times, 7 times, 8 times, 9 times, 10 times, or more. In some embodiments, the solution is exposed to the magnetic field until there is no or substantially no unbound heparin, unbound protamine, unbound ferumoxytol and/or unbound heparin protamine complexes present in the solution.

In some embodiments, the methods further comprise separating the nanoparticles based on size. In some embodiments, the iron content and the strength of the magnetic dipole moment associated with the nanoparticles correlates to the size of the nanoparticles and provides a method to selectively separate the nanoparticles based on size.

In some embodiments, the alternative methods for extracting the nanoparticles from unbound material comprises separating the nanoparticles by ultra-filtration or dialysis. In some embodiments, the heparin, protamine, and an imaging agent are combined in a medium, wherein at least a portion of the heparin, protamine, and an imaging agent complex to form nanoparticles comprising heparin, protamine, and an imaging agent. In some embodiments, the medium is a buffer, serum, a nutrient enriched broth, or a basal medium. In some embodiments, after the nanoparticles are formed, the nanoparticles in the medium are collected from the filtrates to form the preparation comprising the nanoparticles. In some embodiments, following ultra-filtration or dialysis, the medium (i.e., supernatant) is decanted from the preparation comprising the nanoparticles. In some embodiments, the decanted medium comprises unbound heparin, unbound protamine, and/or unbound heparin-protamine complexes.

In some embodiments, exposing the nanoparticles to a magnetic field comprises applying a magnet to the nanoparticles.

In some embodiments, the magnetic field strength is the magnetic flux density of about 300 mT to about 600 mT. In some embodiments, the magnetic flux density is about 300 mT to about 400 mT, about 300 mT to about 500 mT, about 400 mT to about 500 mT, about 400 mT to about 600 mT, or about 500 mT to about 600 mT.

In some embodiments, the applied external magnetic field is uniform and perpendicular to the solution and the collection surface. In certain embodiments, the applied external magnetic field is non-uniform. In some embodiments, separation may be accomplished at the South pole and/or the North pole of a permanent magnet. In yet another embodiment, the separation may also be accomplished at the interface of the two magnets in contact with each other. In another embodiment, separation may be accomplished by applying an applied external magnetic field at a specific strength generated by a current carrying wire in a desired shape.

In some embodiments, the methods selectively separate nanoparticles having iron content of about 0.1 fg per nanoparticle, more than 1,000 atoms encapsulated in the nanoparticle, and the magnetic properties (r1, r2 and r2*) of 3 to 10× greater than of the iron-based imaging agent (e.g., ferumoxytol) by itself. In some embodiments, the methods selectively separate nanoparticles having an iron content greater than about 0.15 fg, about 0.20 fg, about 0.25 fg, about 0.30 fg, about 0.35 fg, about 0.40 fg, about 0.45 fg, about 0.50 fg, about 0.55 fg, about 0.60 fg, about 0.65 fg, about 0.70 fg, about 0.75 fg, about 0.80 fg, about 0.85 fg, about 0.90 fg, about 0.95, or about 1 fg. In some embodiments, the iron content of the nanoparticles is between about 0.1 fg to about 1 fg. For example, in some embodiments, the iron content of the nanoparticles is between about 0.1 fg to about 0.50 fg, about 0.1 fg to about 0.60 fg, about 0.50 fg to about 1 fg, about 0.3 fg to about 0.80 fg, about 0.4 fg to about 0.70 fg, about 0.80 fg to about 1 fg, about 0.1 fg to about 0.4 fg, about 0.1 fg to about 0.65 fg, or about 0.15 fg to about 0.35 fg.

In some embodiments, the methods selectively separate particles having an average diameter about 120 nm to about 195 nm. For example, in some embodiments, the nanoparticles have an average diameter of about 125 nm to about 130 nm, about 125 nm to about 180 nm, about 130 nm to about 160 nm, about 140 nm to about 160 nm, about 140 nm to about 190 nm, about 140 nm to about 150 nm, about 125 nm to about 145 nm, about 130 nm to about 140 nm, about 130 nm to about 150 nm, about 140 nm to about 160 nm, about 140 nm to about 170 nm, about 160 nm to about 175 nm, or about 130 nm to about 180 nm. In some embodiments, the nanoparticles have an average particle size of no more than about 130 nm, no more than about 135 nm, no more than about 140 nm, no more than about 145 nm, no more than about 150 nm, no more than about 155 nm, no more than about 160 nm, no more than about 165 nm, not more than about 170 nm, not more than about 175 nm, no more than about 180 nm, no more than about 190 nm, or no more than about 195 nm.

In some embodiments, the nanoparticles isolated by magnetic separation have an iron loading that is about 30% to about 200% greater relative to nanoparticles that have not been isolated by magnetic separation. In some embodiments, the nanoparticles have an iron loading between about 30% to about 100%, about 50% to about 200%, about 40% to about 80%, about 60% to about 200%, about 50% to about 150%, about 40% to about 120%, about 100% to about 200%, about 150% to about 200%, about 100% to about 180%, about 90% to about 190%, or about 30% to about 60% greater relative to nanoparticles that have not been isolated by magnetic separation.

In some embodiments, the nanoparticles are concentrated at an amount of between about 2× to about 10× without a significant increase in the amount of unbound material. In some embodiments, the nanoparticles, are concentrated to about 0.5×10⁵ particles/mL, about 1.0×10⁵ particles/mL, about 0.5×10⁶ particles/mL, about 1.0×10⁶ particles/mL, about 0.5×10⁷ particles/mL, about 1.0×10⁷ particles/mL, about 0.5×10⁸ particles/mL, about 1.0×10⁸ particles/mL, about 0.5×10⁹ particles/mL, about 1.0×10⁹ particles/mL, about 0.5×10¹⁰ particles/mL, about 1.0×10¹⁰ particles/mL, about 0.5×10¹¹ particles/mL, about 1.0×10¹¹ particles/mL, about 0.5×10¹² particles/mL, about 1.0×10¹² particles/mL, or great. In some embodiments, the particles the nanoparticles are concentrated at an amount of about 1.0×10⁸ to about 1.0×10¹¹ particles/mL. In some embodiments, the nanoparticles are concentrated to a certain amount without a significant increase in the amount of unbound material. In some embodiments, the unbound material includes unbound heparin, unbound protamine, unbound ferumoxytol and/or unbound heparin-protamine complexes.

### Methods of Use

Aspects of the present disclosure relate to methods of labeling a cell comprising contacting the cell with a nanoparticle or a composition comprising nanoparticles. In some embodiments, the methods further comprise labeling a cell-derived product such as a vesicle.

In some embodiments, the methods further comprise labeling the cell under serum starved and/or serum deprived conditions while contacting the cell with the nanoparticle or composition comprising nanoparticles. In some embodiments, the methods comprise labeling the cell under serum starved and/or serum deprived conditions during the contacting step. In some embodiments, the cells remain in the serum starved and/or serum deprived conditions for a certain period of time and then recover for an additional period under normal cell culture conditions.

In some embodiments, the methods include labeling a cell from a cell-derived product, the methods comprising: (a) contacting a first cell with a composition comprising nanoparticles comprised of heparin, protamine, and an iron-based imaging agent, wherein the contacting step labels the cell derived product within the cell; (b) removing the labeled cell derived product from the cell; and (c) contacting a second cell with the cell derived product, wherein the second cell uptakes the labeled cell derived product. In some embodiments, the methods further comprise tracking the second cell.

In some embodiments, the methods further comprise labeling the cells in serum containing media during the contact with the composition. The ability to label the cells without the requirement for serum starvation for a few hours as part of the labeling protocol allows the users to adhere to their existing cell culture Standard Operating Procedures (SOPs) without any substantial change to which the cells are subjected.

In some embodiments, the cell is a mammalian cell, for example, a mouse cell, a rat cell, a canine cell, a porcine cell, a bovine cell, an equine cell, a primate cell, or a human cell.

In some embodiments, the cell is a cancer cell, an immune cell, or a blood cell. In other embodiments, the cell is a stem cell, an embryonic cell, a fetal cell, or a somatic cell. Non-limiting examples of suitable stem cells include a mesenchymal stem cell, a pluripotent stem cell, an induced pluripotent stem cell, an adipose stem cell, a neural stem cell, a T Lymphocyte, a Natural Killer cell or an adult stem cell.

In some embodiments, the methods further comprising tracking the cell for a period of time after contacting the cell with the nanoparticle or composition comprising nanoparticles. In some embodiments, the tracking is performed by imaging the cell. Non-limiting methods for imagining the cell include by fluorescent microscopy, MRI, in vivo optical imaging, PET and/or single-photon emission computed tomography (SPECT), computed tomography (CT) and/or ultrasound.

In some embodiments, the methods further comprise tracking the cell for a period of time using standard MRI imaging sequences. Standard MRI imaging sequences can be used due to the specific magnetic properties of the particles (e.g., clustering effect, high iron load), which enables high efficiency labelling of the.

In some embodiments, the cells are labeled with the nanoparticles or composition comprising nanoparticles in less than about 3 hours with the use of a magnetic labeling system. The magnetic labeling system allows for shortened labeling time by facilitating the rapid uptake of the nanoparticles or composition comprising nanoparticles by application of a magnetic field to the magnetically labeled nanoparticles. In some embodiments, rapid uptake occurs by changing magnetic flux density through the application of permanent magnets or current-driven temporary magnets to the nanoparticles. The force exerted by the magnets facilitates the efficient uptake of the particles to label the cells. For example, in some embodiments, the cells are labeled with the nanoparticles or compositions comprising nanoparticles in less than about 3 hours, less than about 2.5 hours, less than about 2 hours, less than about 1.5 hours, less than about 1 hour, or less than about 0.5 hours.

In some embodiments, the imaging is performed at least about 1 hour, at least about 2 hours, at least about 6 hours, at least about 12 hours, at least about 24 hours, at least about 2 days, at least about one week, at least about a month, at least about 3 months, at least about 6 months, at least about 1 year, at least about 2 years, at least about 3 years, at least about 4 years, at least about 5 years, or more, after the cell is contacted with the nanoparticle or the composition.

In some embodiments, the methods comprising administering a nanoparticle or a composition comprising nanoparticles to a subject. In some embodiments, the subject is a mammalian subject, such as humans, non-human primates or experimental non-human mammalian subjects such as mice or rats. In some embodiments, the mammalian subject is a human.

In some embodiments, the methods comprising administering the nanoparticles or compositions comprising nanoparticles by any appropriate route depending on use and/or the disease or condition to be treated. In some embodiments, the methods comprising administering the nanoparticles or compositions comprising nanoparticles by intravenous, intra-arterial, intramuscular, subcutaneous, intracranial, intranasal or intraperitoneal administration. In some embodiments, the nanoparticle or composition comprising nanoparticles is administered intravenously, intra-arterial, direct tissue injection, and/or intratumorally.

In some embodiments, the methods further comprise programming, genetically modifying and/or tracking the cell for a period of time after administration to the subject. In some embodiments, the tracking is performed by imaging at least a portion of the subject. In some embodiments, the imaging is performed by fluorescent detection, MRI, FL, in vivo optical imaging, PET/SPECT, CT and/or ultrasound.

In some embodiments, the imaging is performed at least about 1 hour, at least about 2 hours, at least about 6 hours, at least about 12 hours, at least about 24 hours, at least about 2 days, at least about one week, at least about a month, at least about 3 months, at least about 6 months, at least about 1 year, at least about 2 years, at least about 3 years, at least about 4 years, at least about 5 years, or more, after the subject is administered with the nanoparticle or the composition.

In some embodiments, the methods of labeling cells allow for imaging by MRI due to the about 30% to about 300% increase in iron loading in the nanoparticles relative to nanoparticles that have not been magnetically separated. In some embodiments, the nanoparticles have an iron loading between about 30% to about 100%, about 50% to about 300%, about 40% to about 80%, about 60% to about 300%, about 50% to about 150%, about 40% to about 120%, about 100% to about 300%, about 150% to about 300%, about 100% to about 180%, about 90% to about 190%, or about 30% to about 60% relative to particles that have not been magnetically separated.

In some embodiments, the methods comprise ex vivo, in vitro, and/or in vivo cell tracking.

In some embodiments, the methods further comprise encapsulating nucleic acids in the nanoparticles before introduction to the cells. Encapsulation of nucleic acids in the nanoparticles provides a safer more effective introduction of the nucleic acids into the cells. The introduction of nucleic acids such as an RNA, plasmid DNA, a DNA or a peptide/protein has been challenging with low efficiency, and has some undesired detrimental effect on the cells using the currently available methods, for example, electroporation and lipofectamine- or a transfection agent-mediated delivery. In some embodiments, the methods further comprise modifying the cells with the nanoparticles having encapsulated nucleic acids. The cells can be modified with the nanoparticles having encapsulated nucleic acids by reprogramming the cells, differentiating the cells, or increasing transfection of the cells.

In some embodiments, the nanoparticles can be used for labeling and tracking of cell-derived biologics such as vesicles to further use them in diagnostic and/or therapeutic applications.

In some embodiments, the nanoparticles can be used for 3-dimensional (3D) printing. In some embodiments, the 3D printing is bioprinting. In some embodiments, the methods include creating 3D scaffolds comprising cells labeled with the nanoparticles for applications ranging from biosensing, tissue regeneration, environment sensing, drug discovery and clinical implementation.

### Examples

The foregoing and the following examples are merely intended to illustrate various embodiments of the present invention. The specific modifications discussed above are not to be construed as limitations on the scope of the invention. It will be apparent to one skilled in the art that various equivalents, changes, and modifications may be made without departing from the scope of the invention, and it is understood that such equivalent embodiments are to be included herein.

There is a need to develop techniques for cell-labeling and tracking via non-invasive MRI detection. Towards this aim, a magnetic separation process has been developed to synthesize and produce a population of herapin (H): protamine (P): feraheme (F) particles (HPF particles) with enriched iron content and increased magnetic properties (VMI NCE 1 nanoparticles) in order to improve the MRI signal for use with conventional MRI sequences available in most clinics. Significantly, the results from this study provide magnetically enriched HPF particles having a high cell-labeling efficiency and a favorable safety profile. The results further demonstrate that the HPF particles produced and manufactured using the methods disclosed herein can be used in cell labeling, cell tracking, biologics labeling, biologics tracking, and cell reprogramming.

### Example 1: SOP for Standard HPF Formation

The objective of the following study was to develop a standard operating procedure (SOP) for standard HPF (Std HPF) formation.

The following regents were used for the preparation of the standard HPF particles: Heparin (H; sodium injection, USP, 1000 Units/ml); Protamine Sulfate (P; injection, USP, 50 mg/ml), and Feraheme (F; injection, USP, 510 mg/17 ml).

To form the standard HPF particles, 2 U/ml solution of H was prepared by first adding a 30 to 500 mL aliquot of a basal medium (e.g., Dulbecco's Modified Eagle Medium/Nutrient Mixture F-12 (DMEM/F-12)) to a sterile tube and/or container. For every 10 mL of the basal medium, a 20 μL of H was added to the sterile tube and/or container comprising the basal medium (e.g., 60 μL of H added to 30 mL of basal medium). The resulting basal medium and H solution was then thoroughly mixed.

As a second step in preparation of the standard HPF, a 60 μL of the P solution was slowly added to each 10 ml of the basal medium containing 2 U/ml H (e.g., 180 μL of P added to 30 mL solution) while swirling the tube effectively during the addition at which point the solution appeared cloudy.

Within approximately 15 to 30 seconds after the P was added, 16.67 μL of the F solution was added to each 10 ml of the above HP solution, and mixed within the tube and/or container (e.g., 50.01 μL of F added to 30 mL of the HP solution).

The procedure as set forth above results in the formation of the Std HPF particles.

### Example 2: SOP for Dual Probe Standard HPF Formation

Because no single imaging modality allows full structural, functional, and molecular information, the combination of two imaging modalities on a single nanoparticle can improve the diagnostic and therapeutic monitoring abilities of the nanoparticles. Accordingly, the objective of the following study was to develop a SOP to generate a dual-labeling agent for multi-modal imaging. Towards this aim, a FITC-conjugated heparin was mixed with protamine and feraheme to form a dual probe standard HPF particles.

To form the dual probe standard HPF particles, a reconstitute of 1 mg of H-FITC was added to 1 mL of sterile water to obtain a solution having a concentration of 70 to 100 U/ml of H-FITC. A 200 μl of the H-FITC solution was then transferred to 10 mL of a basal medium (DMEM/F-12). Following this procedure, an H-FTIC solution having a concentration of 1.4 to 2 U/ml can be obtained.

An aliquot having an appropriate volume of F was then added to a separate sterile tube and/or container and set aside. To prepare the dual probe standard HPF particles, for every 10 mL of basal medium, 16.67 μL of F was required.

Next, an aliquot having an appropriate volume of P was added to a separate tube and/or container. For every 10 ml of a final solution, 60 μL of P is required.

Having separate solutions of each of the components prepared (e.g., H, F, and P solutions), the final step was the preparation of the dual probe HPF particle solution having a concentration ratio of 2 U/ml H-FITC: 60 μg/ml P: 50 μg/ml F. A 60 μL aliquot of the P solution was slowly added to the 10 ml of H-FITC solution. The solution was then mixed (e.g., swirled) at which point the solution appeared cloudy. Approximately within 15 to 30 seconds after the aliquot of the P solution was added, 16.67 μL of the F solution was added and mixed within the tube and/or container.

The procedure as set forth above results in the formation of the Dual probe Std HPF particles.

### Example 3: SOP of the HPF Particles Using Magnetic Separation Process

Having established the SOP for the Std HPF and Dual probe Std HPF particle formation, the next step was to develop an SOP for the production of the HPF particles via magnetic separation process in order to produce/manufacture a population of the HPF particles with enriched iron content, increased magnetic properties and no residual unbound materials (H or P or F or HP); and thereby, improve the overall labeling efficiency of the resulting HPF particles, their safety profile and the MRI activity.

The Std HPF particles formed in Example 1 was transferred to a 15 mL conical tube. Once transferred, a permanent magnet having a magnetic strength of 300 mT to 600 mT was applied to the side of the tube for approximately 25 minutes at room temperature. The separations can be achieved at both the North and South poles of the permanent magnet simultaneously. During the 25 minute application of the magnet, the tube was inverted every 10 minutes. A solid pellet was formed after the 25 minute application of the magnet and the supernatant (i.e., the precipitate-free liquid remaining above the solid material) referred to as the VMI NCE 1 residuals was then transferred to a new tube.

Once the supernatant was transferred, the pellet was washed with an equivalent volume of basal media (DMEM/F-12). Next, a permanent magnet having a magnetic strength of 300 mT to 600 mT was applied again to the side of the tube holding the reconstituted pellet (nanoparticles) for approximately 25 minutes at room temperature in order to extract the HPF particles with enriched iron content and increased magnetic properties. The separations can be achieved at both the North and South poles of the permanent magnet simultaneously. During the 25 minute application of Visicell's magnetic separation system, the tube was inverted every 10 minutes. The supernatant was then removed. Each of the steps were then repeated 3 to 6 times. Similar population of the HPF particles with enriched iron content and increased magnetic properties can be extracted using uniform or non-uniform applied external magnetic field (FIG. 20).

To produce different formulations of the new HPF particles with enriched iron content and increased magnetic properties (ex. different concentrations of particles (different # of particles/ml), an equal volume of the basal medium was added to the pellet to form a solution having 1× the magnetic concentration, the VMI NCE 1 (1×) particles or ⅕ of the volume of basal medium was added to the pellet to form a more concentrated solution having 5× the magnetic concentration, VMI NCE 1 (5×) particles. The same procedure was used for the magnetic separation of the dual-imaging agent, to form Dual probe VMI NCE 1 (1×) and Dual probe VMI NCE 1 (5×) particles.

The procedure as set forth above results in the formation of the VMI NCE 1 (e.g., VMI NCE 1 (1×) and VMI NCE 1 (5×)) particles and Dual probe VMI NCE 1 (e.g., Dual probe VMI NCE 1 (1×) and Dual probe VMI NCE 1 (5×)) particles having an enriched iron content relative to the Std HPF and Dual probe std HPF particles.

### Example 4: Ultra-Filtration Purification: Ultrafiltration Purification

In addition to the development of the magnetic separation process as described above, an ultrafiltration purification process was also developed for the isolation of the HPF particles of increased iron content, desired sizes and reduced unbound materials.

The Std HPF particles formed in Example 1 was transferred to a Corning SpinX filter with 100 Kda MWCO for centrifugation. The Std HPF particles were then centrifuged in a swinging buck centrifuge for 15 to 20 min at 2,000 RPMs or until only a volume of 0.25 mL remained. Following filtration with the Corning SpinX filter, the filtrate referred to as the VMI NCE 2 residuals was transferred into a 15 mL conical tube and the retentate (i.e., the material that does not pass through the filter), referred to as the VMI NCE 2, was washed with an equal volume of basal media. The basal media washed retentate was then re-centrifuged for 15 to 20 min at 2,000 RPM until only 0.25 mL remained. The filtrate was then removed. Each of the steps were repeated 4 to 6 times.

To produce different formulations of the new HPF particles with enriched iron content, desired sizes and reduced unbound materials (ex. different concentrations of particles (different # of particles/ml), an equal volume of the basal medium was added to the VMI NCE 2 to form a solution having 1× the magnetic concentration, the VMI NCE 2 (1×) particles or ⅕ of the volume of basal medium was added to the retentate to form a more concentrated solution having 5× the magnetic concentration, VMI NCE 2 (5×) particles.

The procedure as set forth above results in the formation of the VMI NCE 2 particles.

### Example 5: Extracellular Vesicle (EV) Labeling

Having developed a methodology for isolating HPF particles enriched in iron and increased magnetic properties, the next step was to develop a method for extracellular vesicle labeling with the HPF particles.

Mesenchymal stem cells (MSCs), neural stem cells (NSCs) and Amniotic fluid stem cells (AFSCs) were incubated with any of the HPF particles as described in Examples 1 to 3 for 4 hours in the absence of fetal bovine serum (FBS), followed by an overnight recovery in media containing 10% FBS. The media of the labeled cells was then aspirated. Following aspiration, the cells where washed twice with PBS. EV-depleted media was then added to the cells. The labeled cells were then incubated for 48 to 72 hours.

After incubation, the media (i.e., conditioned media) in which the cells were suspended was collected. The conditioned media was then centrifuged at 2,000×g for 20 minutes in order to remove cellular debris. After centrifugation, the supernatant was then transferred to ultracentrifuge tubes and centrifuged at 120,000×g for approximately 2 hours. After the 2 hours of centrifugation, the supernatant was removed, and the collected EVs were resuspended in PBS. Each of the centrifugation steps were performed at 4° C.

Following the steps outlined above, extracellular vesicles were labeled with the HPF particles in Examples 1 to 3.

### Example 6: Labeling Efficacy, Safety Profile, and Physiochemical Characteristics of HPF Particles

Having isolated and then labeled cells with the HPF particles, next steps included studying the labeling efficacy, the safety profile, and how the physiochemical characteristics of the Std HPF, VMI NCE 1 and Dual probe VMI NCE 1 particles influence the ability to label cells.

### Labeling Efficacy

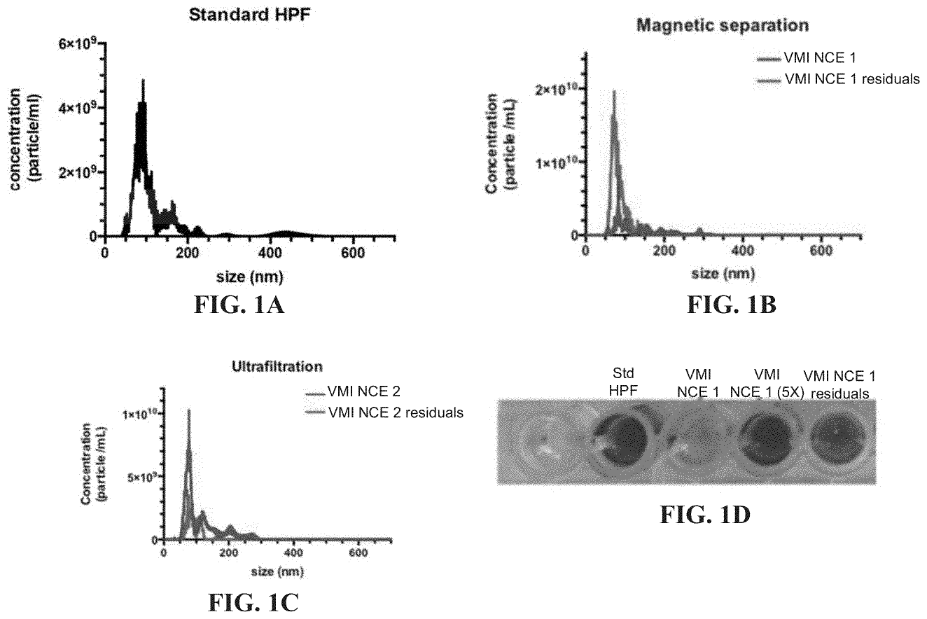

Using Nanoparticle Tracking Analysis (NTA), the particle size distribution of Std HPF and VMI NCE 1 (1×) was compared. The Std HPF prepared as described in Example 1 had a mean diameter of 121 nm. (FIG. 1A, Table 1). In comparison, VMI NCE 1 (1×) prepared as described in Example 3 were larger with mean diameter of 140 nm, while the remaining supernatant from the magnetic separation, VMI NCE 1 residuals, had a mean diameter of 86 nm (FIG. 1B, Table 1). The VMI NCE 2 isolated via the ultrafiltration purification process described in Example 4 had a mean diameter of 117 as compared to the VMI NCE 2 residuals having a mean diameter of 83 nm. (FIG. 1C, Table 1). It is contemplated that the larger iron containing VMI NCE 1 (1×) were enriched during the magnetic separation process, while the unbound H, unbound P, unbound HP and smaller free ferumoxytol were not retained and are concentrated in the VMI NCE 1 residuals fraction as demonstrated in FIG. 1D, resulting in the smaller mean particle diameter for the VMI NCE 1 residuals.

TABLE 1
Diameter of the Resulting HPF Particles Described in Examples 1 to 3.

| | | VMI NCE 1 | VMI NCE 1 | | VMI NCE 2 |
|---|---|---|---|---|---|
| Diameter | Std HPF | (1x) | residuals | VMI NCE 2 | residuals |
| Mean (nm) | 121.4 ± 8.0 | 140.7 ± 5.6 | 86.1 ± 0.7 | 117.2 ± 1.4 | 98.4 ± 10.5 |
| Mode (nm) | 82.8 ± 6.2 | 86.3 ± 6.7 | 75.1 ± 3.4 | 74.2 ± 3.2 | 83.2 ± 5.0 |

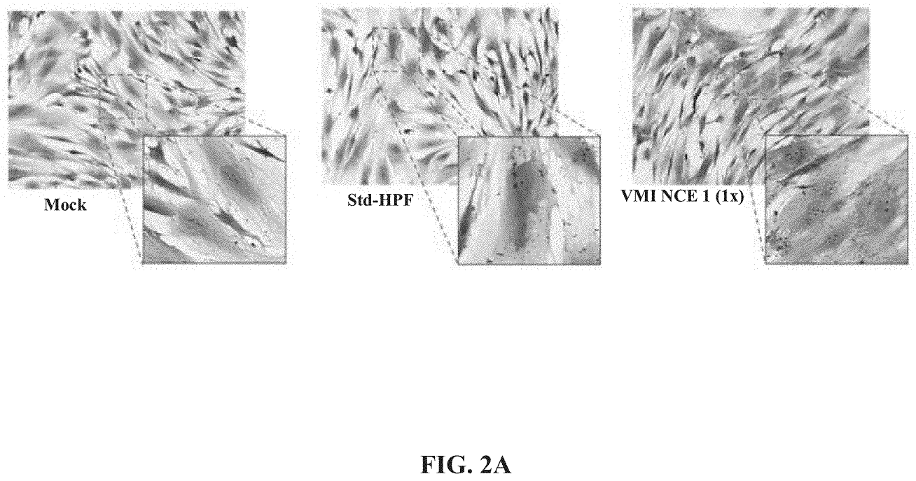

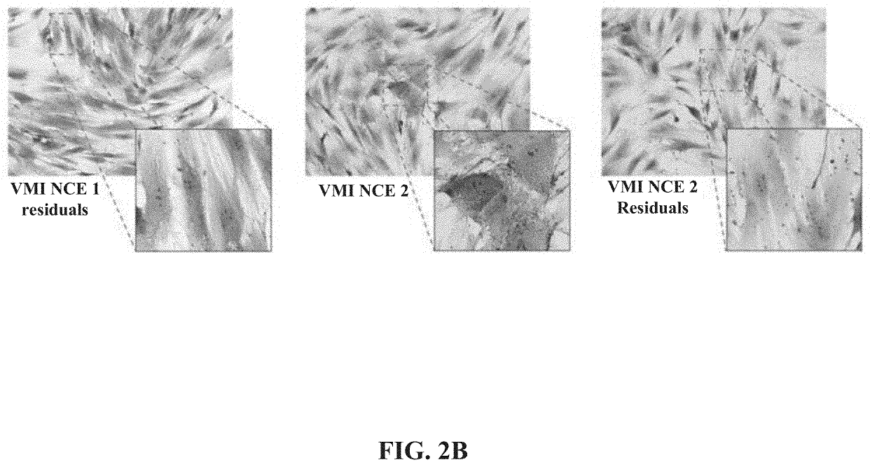

FIGS. 2A-2B demonstrate that HPF particles produced and manufactured using the magnetic separation methodology, have better labeling efficiency and intracellular iron loading as determined by Prussian blue staining (i.e., iron signaling) in low phagocytic cells such as mesenchymal stem cells (MSCs). In particular, FIGS. 2A-2B show the labeling efficiency for unlabeled MSCs (i.e., Mock cells), Std HPF-labeled MSCs, VMI NCE 1 (1×)-labeled MSCs, VMI NCE 1 residuals-labeled MSCs, VMI NCE 2-labeled MSCs, and lastly, the VMI NCE 2 residuals-labeled MSCs. As shown in FIGS. 2A-2B, MSCs labeled with VMI NCE 1 (1×) were more efficiently labeled with iron as compared to MSCs labeled with Std HPF.

Accordingly, this data supports that HPF particles prepared by magnetic separation methodology have a high labeling efficiency as evidenced by the intracellular iron loading.

### Safety Profile

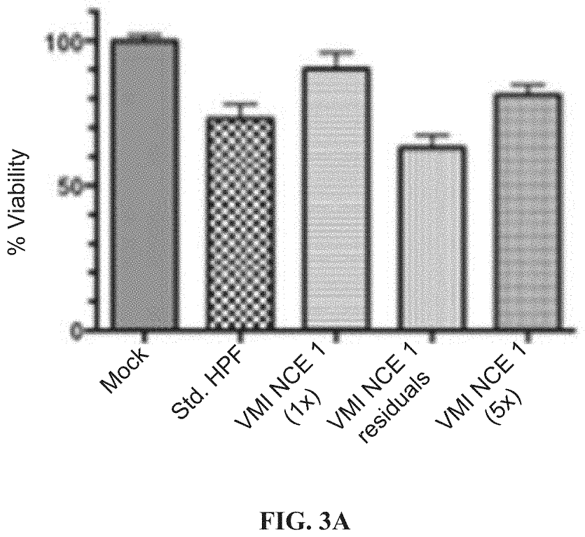

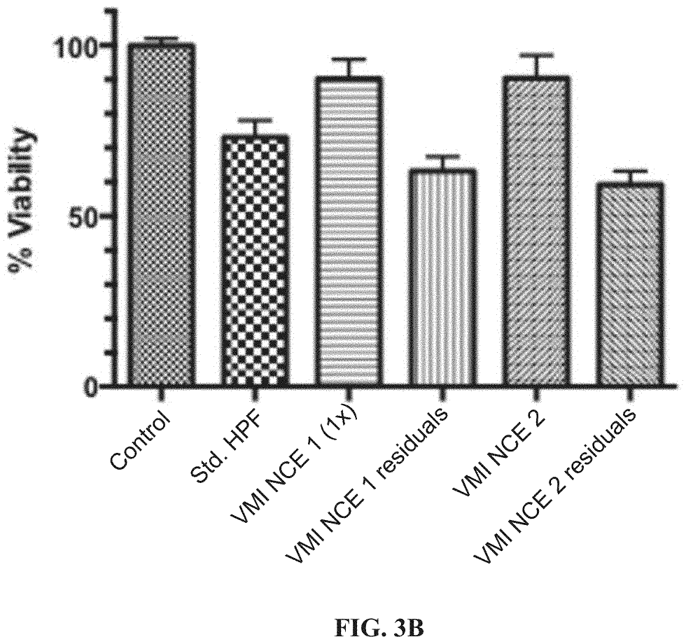

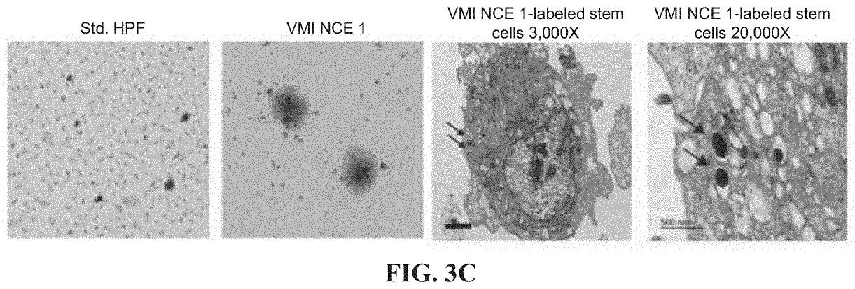

The safety profile of the HPF particles prepared by the methods described in Example 1-4 was then evaluated by labeling the MSCs with those particles and by their mitochondrial dehydrogenase activity. The MSCs labeled with VMI NCE 1 (1×) exhibited minimal toxicity compared to mock-labeled cells as determined by mitochondrial dehydrogenase activity. In contrast, MSC labeled with Std HPF displayed moderate decease in cell viability, while VMI NCE 1 residuals had the most toxic effect on MSCs. (FIG. 3A). The results indicate that the novel VMI NCE 1 (1×) particles possess superior safety profile with about 20% increase in cell viability compared to that of the Std HPF. The results also indicate that the unbound materials such as unbound H, unbound P, unbound HP in the VMI NCE 1 residuals are responsible for the unfavorable effect (40% decrease in viability compared to VMI NCE 1 (1×) and 20% decrease in viability compared to the Std HPF) on the cells. Additionally, it is worth noting that VMI NCE 1 (5×) shows a superior safety profile compared to Std HPF or VMI NCE 1 residuals. Similar results are shown in FIG. 3B, where VMI NCE 1 (1×) and VMI NCE 2 displayed a more favorable safety profile as compared to Std HPF. FIG. 3C additionally demonstrates, that not only are VMI NCE 1 nanoparticles safer, but are also more potent for cell labeling as compared to Std HPF. In particular, FIG. 3C shows by TEM analysis that VMI NCE 1 hold more electron dense magnetically active MRI responsive iron agents relative to Std HPF.

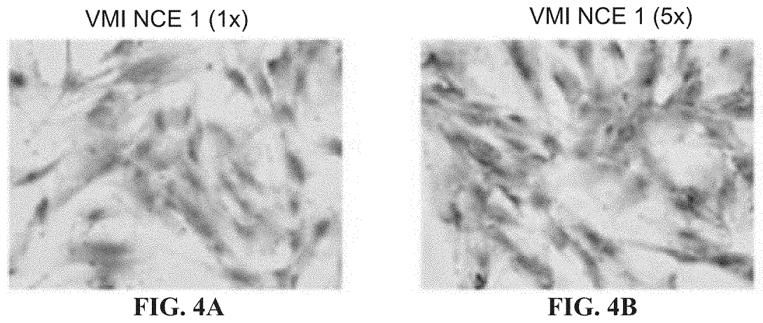

In addition, the total iron concentration of the resulting VMI NCE 1 particles can be tuned by varying the volume of the basal medium added to the pellet after magnetic separation as described in Example 3. In particular, adding an equal volume of basal medium to the pellet after magnetic separation, results in the VMI NCE 1 (1×) particles. However, adding ⅕ of the volume of basal medium to the pellet after magnetic separation, results in a pellet having 5× the magnetic concentration, VMI NCE 1 (5×). MSC labeled with VMI NCE 1 (5×) contained a higher iron content and more intense Prussian blue staining compared to VMI NCE 1 (1×) with only a slight increase in HPF-related toxicity. (FIGS. 4A-4B, respectively). The stability of the HPF particles was also examined and there were no significant changes to labeling efficiency or safety profile after storing the Std HPF or VMI NCE 1 particles at 4° C. for 1 week and at 0° C. for up to 6 months.

As such, and as described above, the HPF particles prepared by magnetic separation have a favorable safety profile and exhibit minimal toxicity towards the labeled cells.

### Physiochemical Composition of HPF Particles

The HPF particles prepared by the magnetic separation methodology where then evaluated to determine if the physiochemical composition of the particles was amenable to a cellular environment.

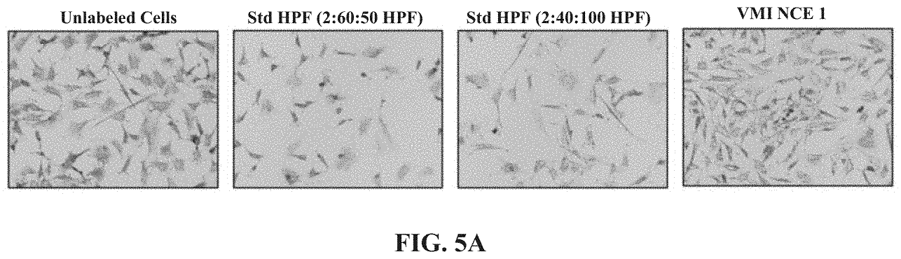

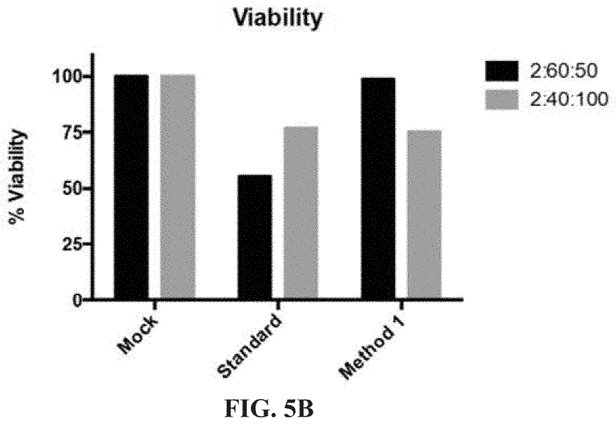

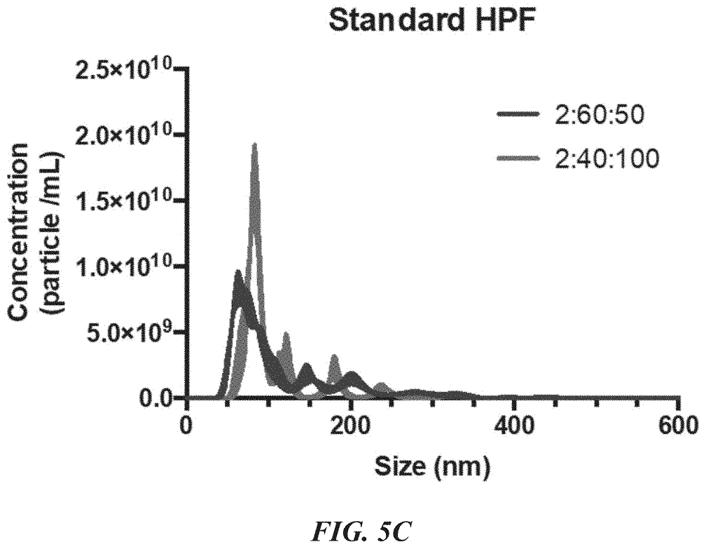

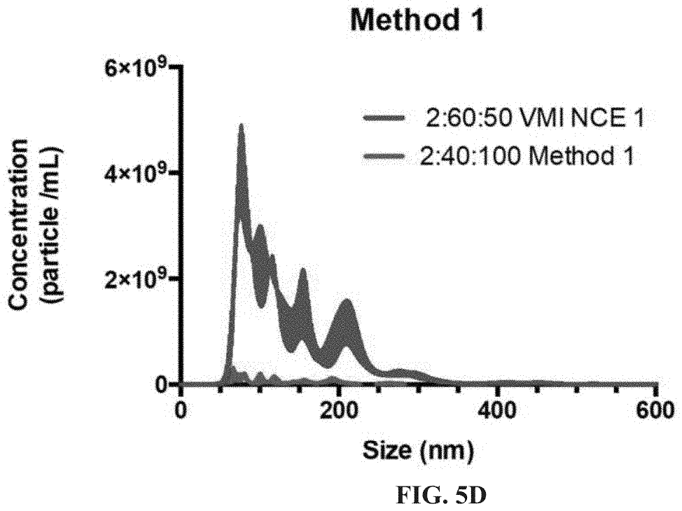

The VMI NCE 1 particles were initially tested for their labeling efficiency within a cellular environment and compared to the Std HPF particles. Std HPF particles were prepared having varied HPF ratios of 2:60:50 HPF and 2:40:100 HPF and VMI NCE 1 particles prepared via the magnetic separation process as described in Examples 1 and 3. FIG. 5A displays images that emphasize the difference in cell labeling efficiency for the Std HPF particles with the variable HPF ratios as compared to VMI NCE 1 particles. As shown in FIG. 5A, the Prussian blue staining for unlabeled cells, Std HPF-labeled cells having a 2:60:50 HPF ratio and Std HPF-labeled cells having a 2:40:100 HPF ratio was less than the VMI NCE 1 labeled cells, suggesting that VMI NCE 1 particles more efficiently label cells as compared to Std HPF particles. FIG. 5B shows the cell viability for the Std HPF-labeled cells having a 2:60:50 HPF ratio, Std HPF-labeled cells having a 2:40:100 HPF ratio, the VMI NCE 1-labeled cells and the cells labeled with 2:40:100 HPF particles produced using the Method 1 (Magnetic separation). FIGS. 5C and 5D show the size distribution for the Std HPF particles and the VMI NCE 1 particles. As enumerated in Table 3, HPF particles enriched with iron during the magnetic separation have a larger particle size.

TABLE 3
Diameter of the Resulting HPF Particles

| | Std HPF | | Std. HPF | 2:40:100 |
|---|---|---|---|---|
| Diameter | 2:60:50 | VMI NCE 1 | 2:40:100 | Method 1 |
| Mean (nm) | 119.9 ± 2.2 | 138.1 ± 2.9 | 91.5 ± 6.0 | 122.8 ± 9.1 |
| Mode (nm) | 66.3 ± 4.1 | 74.5 ± 2.0 | 67.2 ± 2.1 | 58.8 ± 4.3 |

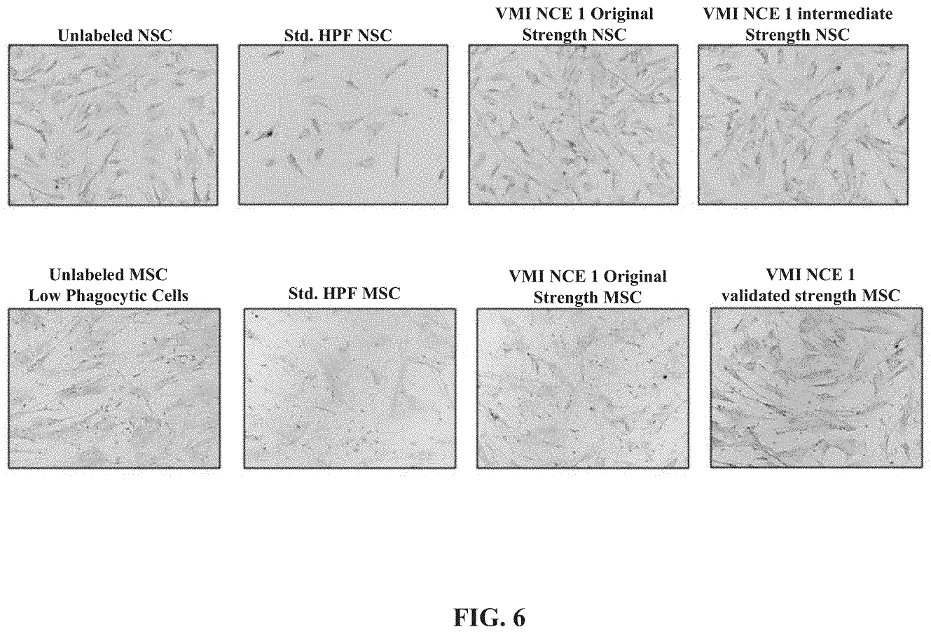

The HPF particles were then evaluated to determine if the total # of particles exposed to the cells, i.e, the total iron content and/or particle concentration could be tuned (i.e., increased) without affecting the cell health. FIG. 6 shows the differential labeling of the disclosed HPF particles in neural stem cells (NSC) (top images) and in low phagocytic cells such as MSC (bottom images). Importantly, FIG. 6 demonstrates that the concentration of the HPF particles introduced into the cells can be increased while maintaining the same degree of cell health. In particular, these results indicate that the HPF particle concentration can be increased 10-fold, providing increased labeling efficiency and iron load without increasing unwanted residuals toxic components in the formulation. Furthermore, there is an evidence of increased intracellular iron loading as shown by the more intense Prussian blue staining without compromising the cell health in each cell type, NSC and MSC, respectively (See FIG. 6, images labeled VMI NCE 1 original strength NSC v. VMI NCE 1 intermediate strength NSC and VMI NCE 1 original strength MSC v. VMI NCE 1 validated strength MSC). Validated strength is 4×-6× for MSC, where 5× usually is a good strength for MSC.

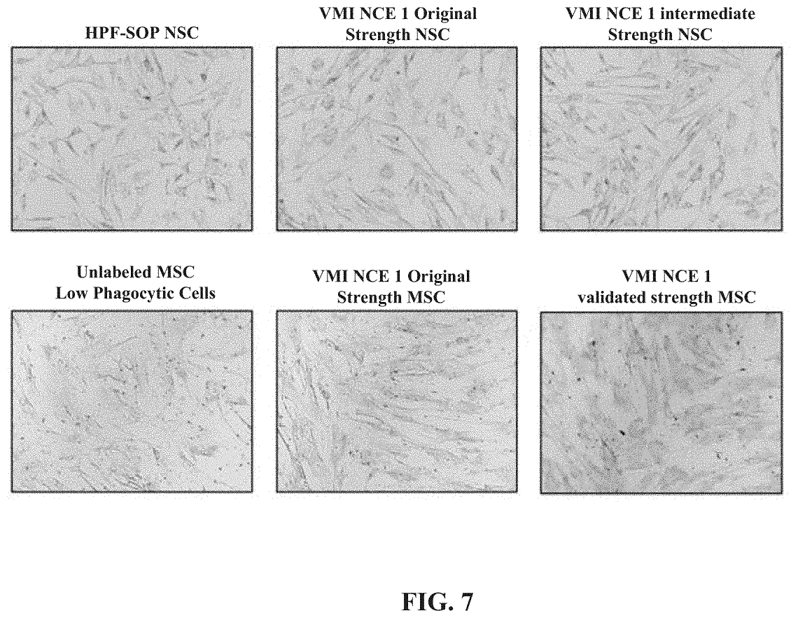

The HPF particles were then tested to determine if they could effectively label cells under serum-containing conditions without the need for serum starvation. This is significant as serum starvation can affect the health of the cells or disrupt the already established SOP and workflow. FIG. 7 shows that not only can the VMI NCE 1 particle concentration be tuned, but also, the particles permit efficient and effective labeling of cells under serum-containing conditions without the need for serum starvation in both cell types, NSC (top images) and MSC (bottom images) compared to Std HPF labeled NSC (top, left most image) and MSC (bottom, right most image).

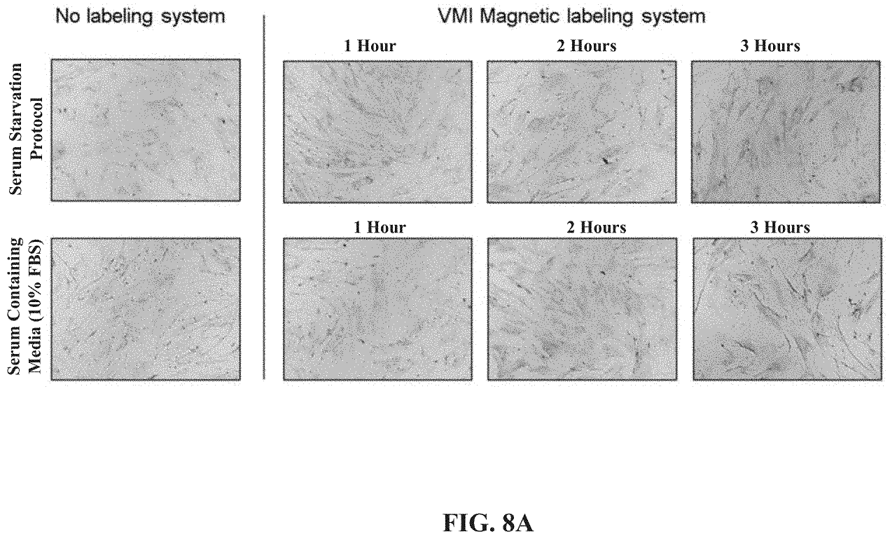

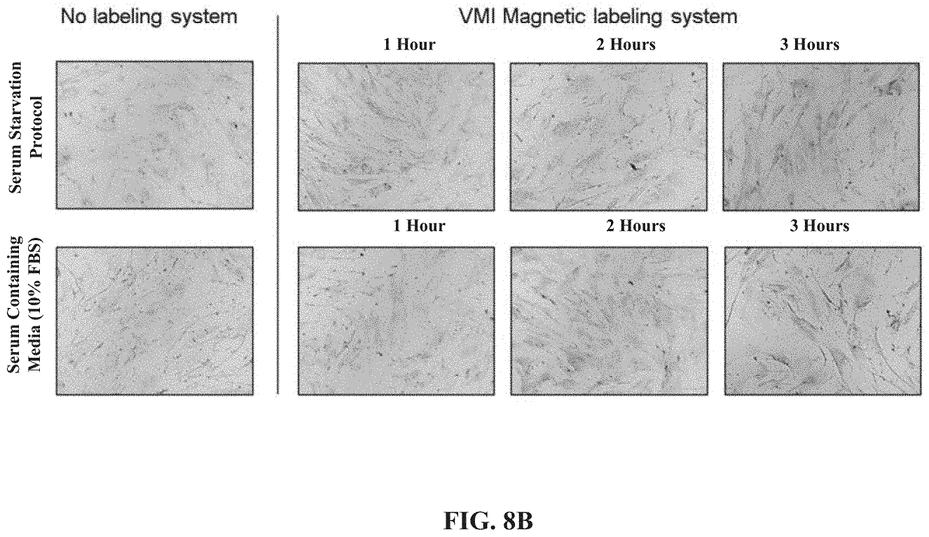

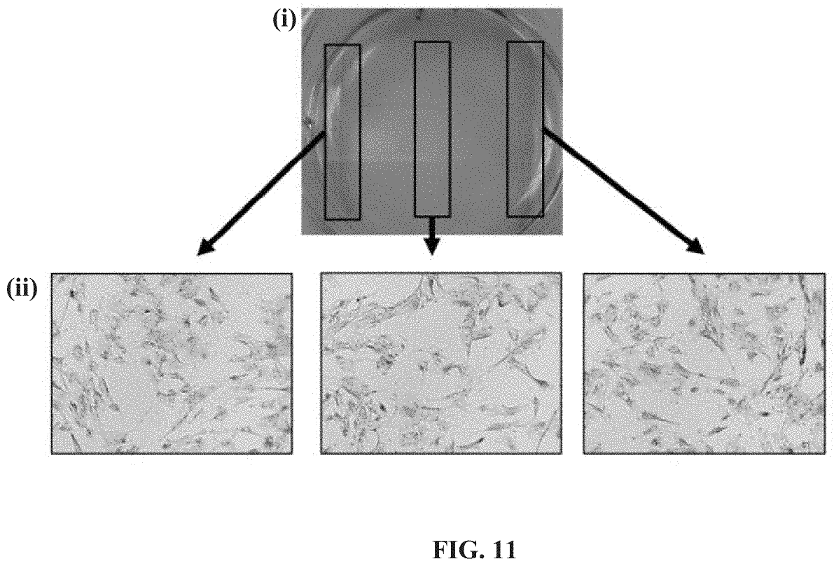

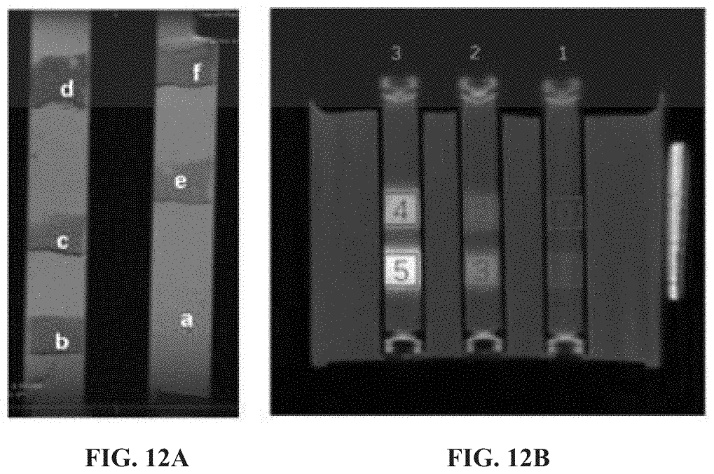

Having determined that the VMI NCE 1 particles label cells effectively under serum-containing conditions overnight, the VMI NCE 1 particles and the VMI magnetic labeling system (FIG. 11) were used in combination to evaluate its ability to increase the rate of labeling (shortened total labeling time) under both serum starved and serum containing conditions. The degree of labeling efficiency was determined for VMI NCE 1 particles under standard serum starvation conditions (FIGS. 8A-8B, top images) as well as under serum containing conditions (FIGS. 8A-8B, bottom images) within a 1 to 3 hour timeframe under each of the conditions in NSC (8A, top and bottom) and in MSC (8B, top and bottom). As shown in FIGS. 8A and 8B, successful labeling at each time point (e.g., 1 hour, 2 hours, and 3 hours) was achieved as indicated by the high intensity of PB staining under both serum starvation conditions and serum containing conditions in both types of cells compared to when no labeling system is used (left most panel (top and bottom) in 8A and 8B).

This study demonstrated that the physiochemical composition of VMI NCE 1 particles prepared by magnetic separation are amenable to high efficiency cell labeling under serum containing conditions. And additionally, when used in combination with the VMI magnetic labeling system, VMI NCE 1 particles allow for shortened labeling time which is suitable for point of care use.

### Example 7: Dual-Imaging VMI NCE 1 Nanoparticles

Having established the efficacy of using the nanoparticles prepared by magnetic separation for cell labeling, the next steps of the study were to further investigate the ability to label cells with the dual probe nanoparticles described in Example 2 and 3.

As described in Example 2 and 3, the dual-imaging agent for multi-modal imaging were prepared by mixing FITC-conjugated Heparin with protamine and feraheme to form the Dual probe Std HPF particles. Following magnetic separation, Dual probe VMI NCE 1 were generated and then used to label MSCs. The Dual probe VMI NCE 1-labeled cells displayed no statistically significant decrease in cell viability as compared to unlabeled cells.

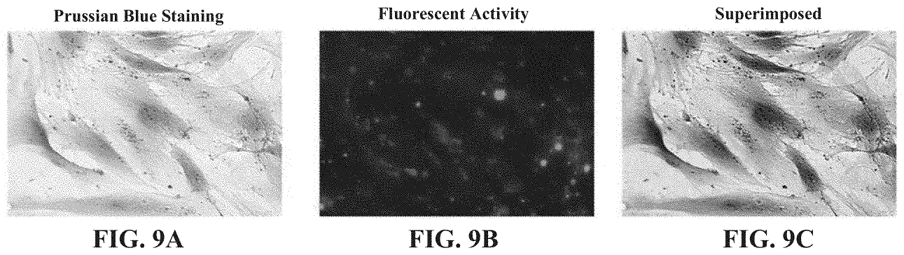

Due to the fluorescent probe (i.e., FITC), the labeling efficiency of Dual probe VMI NCE 1 was evaluated using not only Prussian blue staining to determine the iron loading, but also, via fluorescent activity. (FIGS. 9A-9C). FIG. 9A and FIG. 9B show the Prussian blue staining and the fluorescent activity of the Dual probe VMI NCE 1 particles, respectively and FIG. 9C shows the superimposed images of FIGS. 9A and 9B.

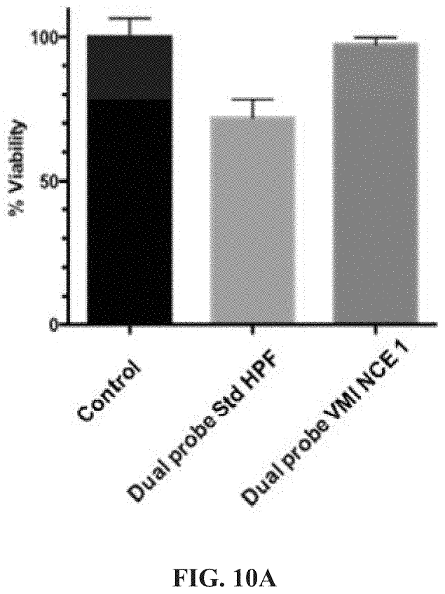

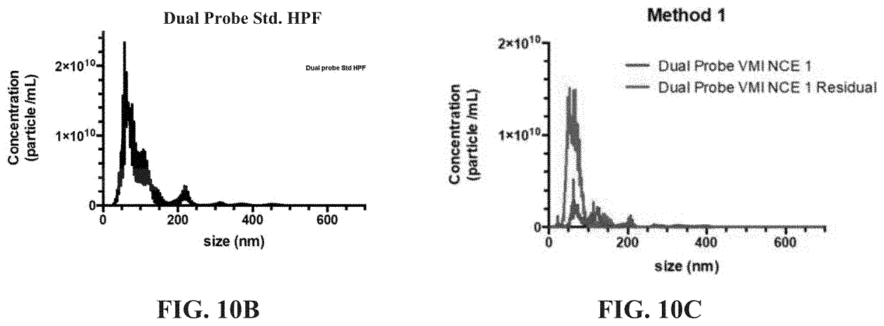

FIG. 10A shows the cell viability of non-labeled cells, Dual probe Std-HPF labeled cells, and the Dual probe VMI NCE 1-labeled cells. As demonstrated in FIG. 10A the viability of the cells labeled with Dual probe VMI NCE 1 particles is superior (about 25% increase in viability) compared to the cells labeled with Dual probe Std HPF particles. FIGS. 10B and 10C show the size distribution for the standard Dual probe Std HPF particles and the Dual probe VMI NCE 1 particles. As enumerated in Table 4, HPF particles enriched with iron during the magnetic separation have a larger particle size.

TABLE 4
Diameter of the Resulting HPF-FITC Particles

| | | | Dual probe |
|---|---|---|---|
| | Dual probe | Dual probe | VMI NCE 1 |
| Diameter | Std HPF | VMI NCE 1 | residuals |
| Mean (nm) | 98.8 ± 12.8 | 121.0 ± 7.7 | 69.1 ± 3.1 |
| Mode (nm) | 66.7 ± 5.8 | 69.9 ± 4.0 | 59.9 ± 4.9 |

Importantly, the data indicates that the Dual probe VMI NCE 1 particles efficiently label cells as evidenced by the Prussian blue staining and fluorescent activity.

### Example 8: MRI Activity of the VMI NCE 1 Nanoparticles

The objective of the following study was to determine the performance of the VMI NCE 1 particles for magnetic resonance imaging (MRI).

A representative drawing of a magnetic labeling system that allows for shorter labeling time (e.g., less than 3 h) by facilitating rapid uptake of the VMI NCE 1 particles produced and manufactured using the method disclosed herein is shown in FIG. 11. The VMI magnetic labeling system employs the change in magnetic flux density produced by permanent magnets or current-driven temporary magnets (electromagnets) to exert a force on the VMI NCE 1 magnetic particles, which through the interaction, causes the deflection of the particles from its original positions in suspension and helps facilitate the uptake of those nanoparticles to efficiently and effectively label the cells at the desired iron load in a shortened time frame of less than 3 hours. The change in magnetic flux density (directional and/or magnitude with the magnitude greater than 400 mT) can be exposed to the desired areas of the cells for selectively labeling the cells in those areas. The cells should be positioned between 0.00 mm to 5 mm from the surface of the applied magnets. In addition, the particle collection surface should be within 0.00 mm to 4 mm from the surface of the applied magnets for the magnetic separation process. FIG. 11 shows that a change in magnetic flux density by permanent magnets or current-driven temporary magnets is used to facilitate the uptake of the VMI NCE 1 particles to efficiently and effectively label the cells for both at a desired percentage of cells and iron load per cell (See FIG. 11 step (i)). FIG. 11 step (ii) shows images of particle accumulation and labeled cells at the desired areas of labeling induced by the VMI magnetic labeling system. The Prussian blue stains in the images show the intensity of labeling with exposure to the appropriate concentration of VMI NCE 1 particles for the desired cell type while using the VMI magnetic labeling system.

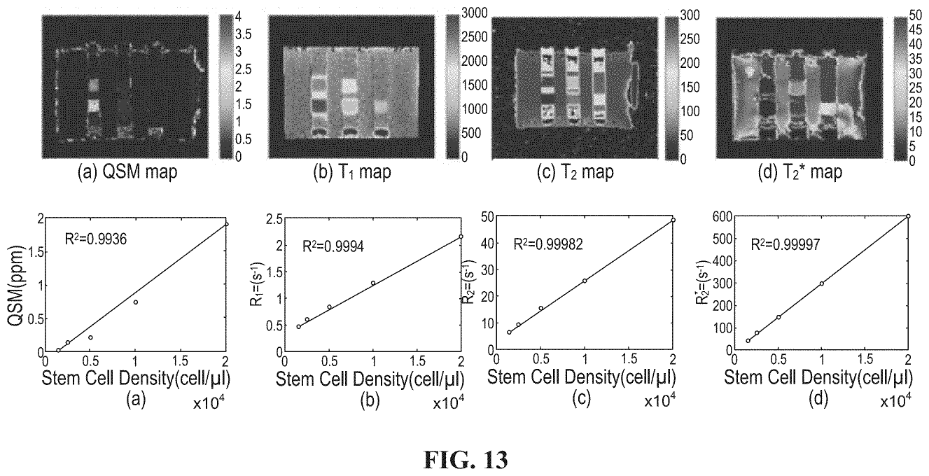

The VMI NCE 1 particles were then evaluated for their ability to provide enhanced signaling via MRI compare to the Std HPF particles. MRI activity was measured by an animal MRI scanner (7T Bruker PharmaScan) as well as clinical MRI scanner (3T GE-MR750) using conventional MRI sequences and quantitative MRI sequences. As shown in FIG. 12A, in vitro T₂ and T₂* MRI signals were determined for (a) non-labeled cells, (b) standard Std HPF-labeled cells, (c) VMI NCE 1-labeled cells at original strength (1×), (d) VMI NCE 1-labeled cells at a validated strength for low phagocytic MSC, (e) dual probe std HPF-labeled cells, and (f) dual probe VMI NCE 1-labeled cells. FIG. 12B, shows a representative T₂* weighted image of the clinical scanner (phantom at 3T GE-MR750). In FIG. 12B, layer 0 was non-labeled stem cells and layers 1 to 5 were the magnetically labeled cells at concentrations of 1500 cells/ul, 2500 cells/ul, 5000 cells/ul, 10,000 cells/ul, and 20,000 cells/ul. FIG. 13 shows the color maps of different MR parameters (e.g., (a) QSM map (b) T₁ map (c) T₂ map (d) T₂* map) (top images); and their correlation fittings between the density and the MR parameters (lower images). Lastly, Table 1 enumerates the quantitative MR results of the non-labeled, and VMI NCE 1-labeled stem cells at different densities.

| | Number | | | | | |
|---|---|---|---|---|---|---|
| | 1 | 2 | 3 | 4 | 5 | 6 |
| Density (cell/mL) | 5000 | 1500 | 2500 | 5000 | 10000 | 20000 |
| Volume (mL) | 0.5 | 0.5 | 0.5 | 0.5 | 0.5 | 0.5 |
| T₁ (ms) | 2516.42 ± 64.34 | 2124.24 ± 74.16 | 1655.37 ± 41.43 | 1187 ± 48.30 | 776.30 ± 24.73 | 463.53 ± 31.15 |
| T₂ (ms) | 271.04 ± 4.77 | 160.73 ± 1.93 | 109.38 ± 0.71 | 66.61 ± 1.20 | 38.73 ± 0.92 | 20.58 ± 0.76 |
| T₂* (ms) | 496.55 ± 68.42 | 27.29 ± 0.70 | 14.04 ± 0.27 | 6.94 ± 0.30 | 3.41 ± 0.16 | 1.67 ± 0.17 |
| QSM | 0.02 ± 0.02 | 0.03 ± 0.03 | 0.14 ± 0.03 | 0.21 ± 0.07 | 0.74 ± 0.11 | 1.91 ± 0.76 |

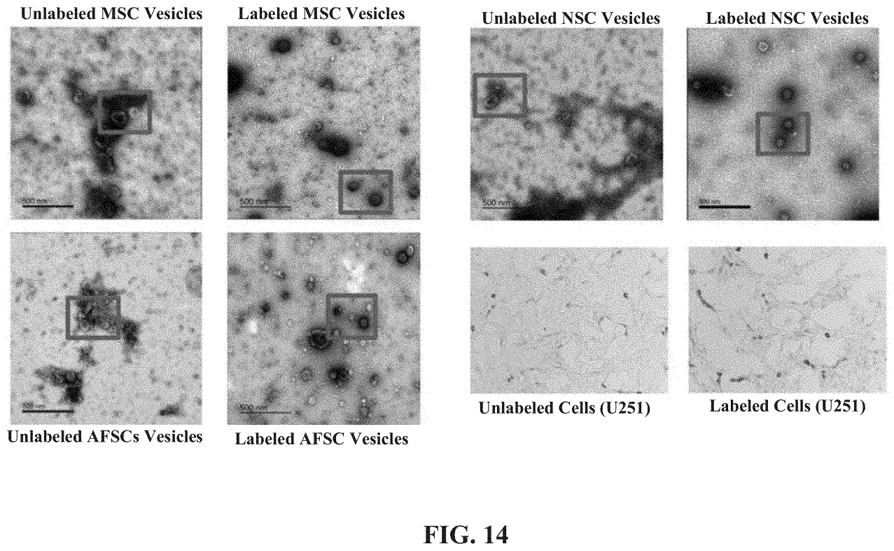

Having determined that the VMI NCE 1 particles have a favorable magnetic profiles, the particles were then evaluated to determine if they could provide in vivo tracking of cellular-derived particles. FIG. 14 shows TEM images of HPF particle labeling of cellular derived particles, which were vesicles derived from NSC, MSC, and amniotic fluid stem cells (AFSC). As shown in FIG. 14, TEM analysis of unlabeled vesicles reveal that the unlabeled vesicles have a hollow disk-like shape. In contrast, labeled vesicles appear as round and has having a high electron density. Both the unlabeled and labeled vesicles were introduced to a glioblastoma cell line (U251) to determine if the cells would uptake the labeled vesicles. Significantly, and as shown in FIG. 14, the cells efficiently up took the labeled vesicles as evidenced by the high iron loading inside the cells via Prussian blue staining. (FIG. 14, bottom right images).

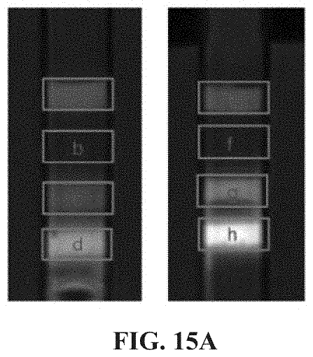

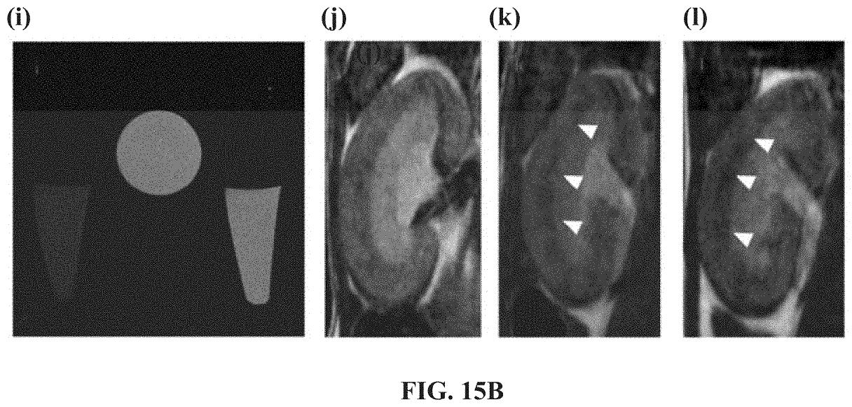

The VMI NCE 1 particle labeling of cellular-derived particles was further investigated by MRI activity in the diseased kidney of a mouse. FIG. 15A shows the VMI NCE 1-labeled vesicles in a clinical MRI scanner (3T GE-MR750) using a T2*-weighted conventional MRI sequence and FIG. 15B shows the VMI NCE 1-labeled vesicles in a T2 weighted image of the diseased kidney at 7T Burker Pharmascan.

In FIG. 15A, the difference in contrast-enhanced signal intensities, and the increase in signal intensities with the increase of the densities of the VMI NCE 1-labeled vesicles can be observed. FIG. 15A shows VMI NCE 1 particles (positive controls) in (a) and (e); 2.5×10¹⁰ non-labeled vesicles (negative controls) from NSC and AFSC (b) and (f), respectively; 2.5×10¹⁰ labeled vesicles from NSC and AFSC (c) and (g) respectively, and lastly, 1×10¹¹ labeled vesicles from NSC and AFSC (d) and (h), respectively.

In FIG. 15B, (i) shows the phantom images of VMI NCE 1-labeled vesicles (left tube), the reference (center, round) and the non-labeled vesicles (right tube) and the right images show the left kidney of a mouse (j) before injection of the VMI NCE 1-labeled vesicles, (k) 20 minutes after injection of the VMI NCE 1-labeled vesicles and (l) 3 hours after injection of the VMI NCE 1-labeled vesicles. The white arrows in images labeled (k) and (l) show the hypointense signals that indicate the presence of VMI NCE 1-labeled vesicles in the diseased kidney.

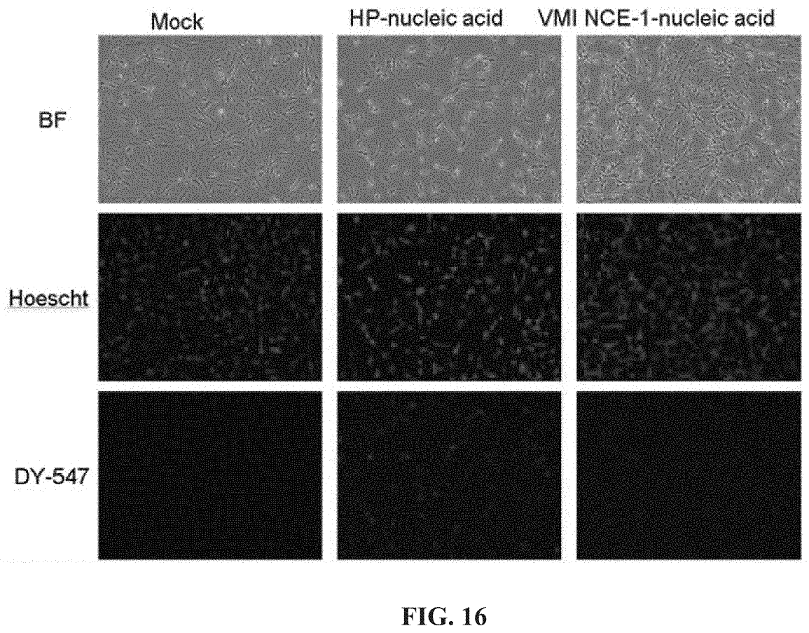

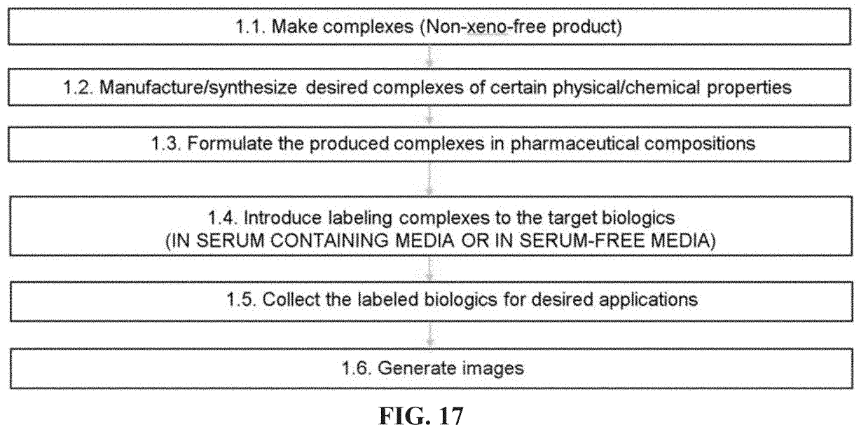

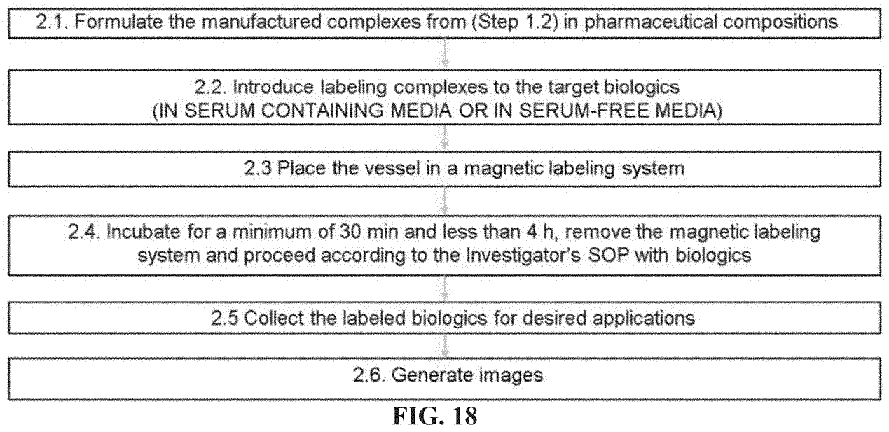

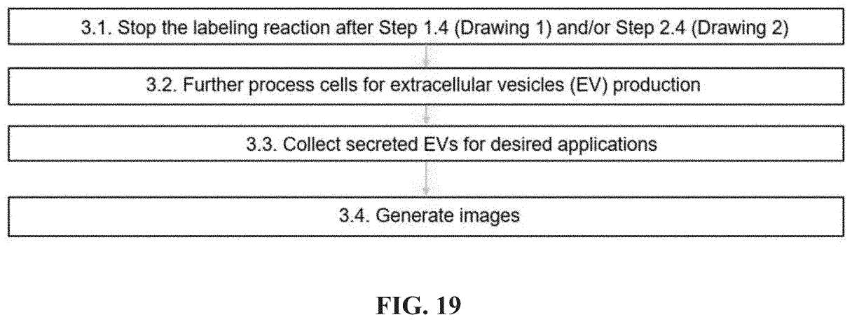

Lastly, the VMI NCE 1 particles were evaluated to determine if they would allow encapsulation of nucleic acid cargos inside the particles for a safer more effective introduction of such nucleic acids to the cells for therapeutic or reprogramming applications. To evaluate the VMI NCE 1 particles ability to encapsulate nucleic acids, bright field (BF) and fluorescent (i.e., Hoescht (nucleus) and DY-547-nucleic acid) assays were run on non-labeled NSCs (Mock), NSCs labeled with a control particles carrying nucleic acid with known toxic effect on the cells due to residual H or P components, and NSCs labeled with VMI NCE 1 particles carrying nucleic acid. As shown in FIG. 16, the physiochemical composition of the VMI NCE 1 particles allows for the efficient encapsulation of nucleic acids, providing a more effective and safer method for introducing the nucleic acids into stem cells.

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

Specifically intended to be within the scope of the present invention, and incorporated herein by reference in its entirety, is the following publication: Thu M S, Bryant L H, Coppola T, Jordan E K, Budde M D, Lewis B K, Chaudhry A, Ren J, Varma N R, Arbab A S, Frank J A. Self-assembling nanocomplexes by combining ferumoxytol, heparin and protamine for cell tracking by magnetic resonance imaging. Nat Med. 2012; 18(3):463-7. Epub 2012/03/01. doi: 10.1038/nm.2666. PubMed PMID: 22366951; PMCID: PMC3296876.

1. A nanoparticle comprising heparin, protamine, an iron-based imaging agent, and a contrast agent, wherein the contrast agent is conjugated to the heparin and/or the protamine.

2. The nanoparticle of claim 1, wherein the nanoparticle has an iron content of about 0.1 femtograms (fg) to about 1 fg.

3. The nanoparticle of claim 1, wherein the iron-based imaging agent is ferumoxytol.

4. The nanoparticle of claim 1, wherein the contrast agent is a fluorescent probe, a PET agent, a CT agent, or an MM agent.

5. The nanoparticles of claim 1, wherein the nanoparticles are uniform in size and have an average diameter of 120 nm to 195 nm.

6. A composition comprising nanoparticles comprised of heparin, protamine, and an iron-based imaging agent, and wherein the composition comprises no or substantially no unbound heparin, protamine, or iron-based imaging agent.

7. The composition of claim 6, wherein the composition comprises no or substantially no unbound heparin-protamine complexes, protamine-iron-based imaging agent complexes, or heparin-iron-based imaging agent complexes.

8. The composition of claim 6, further comprising an excipient.

9. The composition of claim 6, wherein the composition is lyophilized or frozen.

10-44. (canceled)
